SEQUENCE LISTING (6 sequences)

SEQ ID NO: 1 - DNA, 35 bp, Artificial Sequence
  synthesized
gatacaatatgttccaaaaaggaacatattgtatc

SEQ ID NO: 2 - DNA, 35 bp, Artificial Sequence
  synthesized
agaacatgctgttccaaaaaggaacagcatgttct

SEQ ID NO: 3 - DNA, 21 bp, Artificial Sequence
  synthesized
cgnnnnncaaaaagnnnnncg

SEQ ID NO: 4 - DNA, 21 bp, Artificial Sequence
  synthesized
cgatgcacaaaaagtgcatcg

SEQ ID NO: 5 - DNA, 21 bp, Artificial Sequence
  synthesized
cgtgcatcaaaaagatgcacg

SEQ ID NO: 6 - DNA, 21 bp, Artificial Sequence
  synthesized
cgaaattcaaaaagaatttcg

(19) United States
(12) Patent Application Publication
(10) Pub. No.: US 2003/0165837 A1
(43) Pub. Date: Sep. 4, 2003

(54) Method for screening DNA binding

(75) Inventor: Dale L. Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 09/866,077

(22) Filed: May 25, 2001

Related U.S. Application Data
(60) Provisional application No. 60/211,760, filed on Jun. 14, 2000.

(51) Int. Cl. C12Q 1/68

(52) U.S. Cl. 435/6

(57) ABSTRACT

A simple, nondestructive, and high throughput method for establishing DNA binding affinity and sequence selectivity is based on the loss of fluorescence derived from the displacement of ethidium bromide or thiazole orange from the DNA of interest or, in selected instances, the change in intrinsic fluorescence of a DNA binding agent itself. The method is applicable for assessing relative or absolute DNA binding affinities. Enlisting a library of hairpin deoxyoligonucleotides containing all 5 base pair (512 hairpins) or 4 base pair (136 hairpins) sequences displayed in a 96-well format, a compound's rank order binding to all possible sequences is generated resulting in a high resolution definition of its sequence selectivity using this fluorescent intercalator displacement (FID) assay. As such, the technique complements the use of footprinting or affinity cleavage for the establishment of DNA binding selectivity and provides the information at a higher resolution. The merged bar graphs generated by this rank order binding provide a qualitative way to compare, or profile, DNA binding affinity and selectivity.

### CROSS-REFERENCE TO RELATED APPLICATION

The present application is a Non-Provisional Application of and claims priority, under 35 U.S.C. §119(e), to provisional application Serial No. 60/211,760, filed Jun. 14, 2000, the disclosure of which is hereby incorporated by reference.

### GOVERNMENT RIGHTS

This invention was made, in part, with government support under Grants from NIH, viz., Grants No. CA 41986 and CA78045. The U.S. government may have certain rights in the invention.

### SEQUENCE LISTING

A sequence listing is appended hereto.

### TECHNICAL FIELD

The invention relates to methods, compositions, and articles employable for determining DNA binding affinity and sequence selectivity of molecules. More particularly, the invention relates to methods, compositions, and articles that employ fluorescent intercalator displacement for determining DNA binding affinity and sequence selectivity of molecules.

### BACKGROUND

A variety of techniques are commonly used to investigate the DNA binding properties of small molecules (Fox, K. R., Ed., Drug-DNA Interactions Protocols; Methods in Molecular Biology; Humana Press: Totowa, N.J. 1997; Vol. 90; and Jenkins, T. C. Optical Absorbance and Fluorescence Techniques for Measuring DNA-Drug Interactions, In Drug-DNA Interactions Protocols; Fox, K. R. Ed.; Methods in Molecular Biology; Humana Press: Totowa, N.J. 1997; Vol. 90, p 195). However, most approaches are technically challenging and time consuming, rendering them inapplicable to high-throughput screening. The commonly used DNAse I footprinting is able to give base pair resolution of preferred DNA binding sequences, but suffers from the sequence specificity of DNAse I itself (Drew, H. R.; Travers, A. A. Cell 1984, 37, 491). Methods such as MPE footprinting and affinity cleavage overcome this problem, but require precisely controlled reaction conditions or the derivatization of the DNA binding agents themselves (Van Dyke, M. W.; Hertzberg, R. P.; Dervan, P. B. Proc. Natl. Acad. Sci. USA 1982, 79, 5470). The recently reported RESPC technique also alleviates some of the commonly encountered problems in footprinting, but requires the preparation of multiple DNA constructs (Hardenbol, P.; Wang, J. C.; Van Dyke, M. W. Bioconjugate Chem. 1997, 8, 616). Most techniques require the knowledge of specialized biochemical procedures and assay reproducibility comes only with experience. None are applicable to high-throughput screening. Two hairpin oligonucleotides employed in the prior art for ascertaining DNA binding are illustrated in FIG. 2. The two hairpin oligonucleotides are related sequences of the androgen response element, i.e., the 14 base pair ARE-consensus sequence (SEQ ID NO:1)(Cato, A. C. B.; Hernerson, D.; Ponta, H. EMBO J. 1987, 33, 545) and the 14 base pair PSA-ARE-3 sequence (SEQ ID NO:2)(Cleutjens, K. B. J. M.; van der Dorput, H. A. G. M.; van Eekelen, C. C. E. M.; van Rooij, H. C. J.; Faber, P. W.; Trapman, J. Mol. Endocrinology 1993, 7, 23).

### SUMMARY

A technically non-demanding method for establishing DNA binding selectivity and affinity is disclosed. In a preferred example of the invention, the technique entails the measurement of the loss of fluorescence derived from the displacement of ethidium bromide or thiazole orange from hairpin deoxyoligonucleotides containing all possible five (512 hairpins) or four bp sequences (136 hairpins) displayed in a 96-well format. In this example, the process of the invention provides a rank order binding to all possible five or four bp sequences resulting in a high resolution definition of a compound's sequence selectivity. The process may also be employed with larger hairpin deoxyoligonucleotide libraries, i.e., libraries containing all possible 6-14 base pair (bp) sequences, although the logistics of these larger libraries becomes increasingly difficult. Alternatively, partial hairpin deoxyoligonucleotide libraries may also be employed.

Herein, technical issues associated with the implementation of the assay are described; parameters affecting the accuracy of the results are defined; experimental conditions suitable for conducting assays applicable to multiple compounds of varying affinities are disclosed; and its use in comparing the DNA binding properties of distamycin A, netropsin, DAPI, Hoechst 33258, and berenil are presented. The full set of 512 hairpin deoxyoligonucleotides may be purchased from GenBase, 6450 Lusk Blvd, Suite E 107, San Diego, Calif. 92121, telephone: 858-453-8879, e-mail genbase@aol.com. Paramount to the success of the approach is the establishment of the hairpin concentration as single strand DNA (80-95° C., UV). Notable observations include the fact that intrinsic fluorescence properties of the DNA binding agents themselves do not interfere with the assay, and if they do, alternative fluorescent intercalators are available for use (TO vs EtBr).

Just as importantly, the assay may be utilized to establish DNA binding constants for a given sequence through quantitative titration. Several methods of establishing binding constants were examined and the most reliable entails a Scatchard or curve fitting analysis of the titration binding curve which also determines the stoichiometry of binding (Brown, K. A.; et al. J. Am. Chem. Soc. 1993, 115, 7072; Satz, A. L.; Bruice, T. C. Bioorg. Med. Chem. 2000, 8, 1871; Satz, A. L.; Buice, T. C. J. Am. Chem. Soc. 2001, 123, 2469. For additional discussions of multiple equilibria or multiple binding modes and the limitations of a Scatchard analysis, see: Norby, J. G.; et al. Anal. Biochem. 1980, 102, 318; Feldman, H. A. Anal. Biochem. 1972, 48, 317; Deranleau, D. A. J. Am. Chem. Soc. 1969, 91, 4050; Glasel, J. A.; et al. J. Biol. Chem. 1976, 251, 2929).

Finally, the technique is nondestructive suggesting that immobilization of the hairpins (chips, beads, glass slides) would permit their rinsing and reuse in subsequent assays. This hairpin immobilization and reuse would remove the barrier to examining complete libraries of longer sequences (>5 base pairs) and extend the use of the technology to the characterization of binding site sizes typical of proteins.

One aspect of the invention is directed to a process for determining DNA binding affinity and sequence selectivity of a test molecule. In the first step of the process, the fluorescent intercalator displacement of the test molecule is measured with respect to each DNA binding reagent within an array of DNA binding reagents. The array of DNA binding reagents includes a plurality of hairpin deoxyoligonucleotides dissolved in an aqueous solvent together with a fluorescent DNA intercalator. Each hairpin deoxyoligonucleotide within said plurality is physically separate from all other hairpin deoxyoligonucleotides within said plurality. The fluorescent DNA intercalator is of a type which is displaceable from said hairpin deoxyoligonucleotides by the test molecule. Measurement of the fluorescent intercalator displacement by the test molecule may be made by detecting and recording fluorescence for each DNA binding reagent within the array of DNA binding reagents; then combining an aliquot of the test molecule with each DNA binding reagent; and then detecting and recording fluorescence a second time for the array of DNA binding reagents for determining fluorescent intercalator displacement. In the second Step of the process, the fluorescent intercalator displacement by the test molecule is then correlated within the array of DNA bind reagents for determining the DNA binding affinity and sequence selectivity of the test molecule. In a preferred mode of this aspect of the invention, each hairpin deoxyoligonucleotide includes a single strand hairpin section and a double strand section. The single strand hairpin section consists of a non-variable sequence of unpaired deoxynucleotide bases. The double strand section includes a variable sequence of paired deoxynucleotide bases. The variable sequence has a set length of between 4 and 14 deoxynucleotide bases. The variable sequence is known or identifiable. The double strand section is attached to the single strand hairpin section. The plurality of hairpin deoxyoligonucleotides includes substantially all possible sequences within the set length of the variable sequence. Preferred fluorescent DNA intercalators include ethidium bromide and thiazole orange. In a preferred mode, the array of DNA binding reagents is contained by an array of microtiter wells. If the set length is 4, then the plurality of hairpin deoxyoligonucleotides includes 136 hairpin deoxyoligonucleotides. If the set length is 5, then the plurality of hairpin deoxyoligonucleotides includes 512 isolated hairpin deoxyoligonucleotides.

Another aspect of the invention is directed to a library employable for assaying DNA binding affinity and sequence selectivity. The library comprises a plurality of hairpin deoxyoligonucleotides. Each hairpin deoxyoligonucleotide includes a single strand hairpin section and a double strand section. The single strand hairpin section consists of a non-variable sequence of unpaired deoxynucleotide bases. The double strand section includes a variable sequence of paired deoxynucleotide bases. The variable sequence has a set length of between 4 and 14 deoxynucleotide bases. The variable sequence is either known or identifiable. The double strand section is attached to the single strand hairpin section. The plurality of hairpin deoxyoligonucleotides includes substantially all possible sequences within the set length of the variable sequence. Each hairpin deoxyoligonucleotide within the plurality is physically separate from all other hairpin deoxyoligonucleotides within such said plurality. In a preferred embodiment of this aspect of the invention, the set length is 4 and plurality of hairpin deoxyoligonucleotides includes 136 hairpin deoxyoligonucleotides. In another embodiment, the set length is 5 and the plurality of hairpin deoxyoligonucleotides includes 512 isolated hairpin deoxyoligonucleotides.

Another aspect of the invention is directed to an array of DNA binding reagents for determining DNA binding affinity and sequence selectivity of a test molecule. The array comprises a plurality of hairpin deoxyoligonucleotides and an aqueous solvent employable for use in a fluorescent intercalator displacement assay. The hairpin deoxyoligonucleotides are dissolved in the aqueous solvent and have a concentration sufficient for use in the fluorescent intercalator displacement assay. Each hairpin deoxyoligonucleotide within the plurality is physically separate from all other hairpin deoxyoligonucleotides within such plurality. Each hairpin deoxyoligonucleotide includes a single strand hairpin section and a double strand section. The single strand hairpin section consists of a non-variable sequence of unpaired deoxynucleotide bases. The double strand section includes a variable sequence of paired deoxynucleotide bases. The variable sequence has a set length of between 4 and 14 deoxynucleotide bases. The variable sequence is known or identifiable. The double strand section is attached to the single strand hairpin section. The plurality of hairpin deoxyoligonucleotides includes substantially all possible sequences within the set length of the variable sequence. In a further embodiment of this aspect of the invention, the array further comprises a fluorescent DNA intercalator. The fluorescent DNA intercalator is intercalated into the hairpin deoxyoligonucleotides and is potentially displacable from the hairpin deoxyoligonucleotides by the test molecule. The fluorescent DNA intercalator is of a type that has altered fluorescent properties upon displacement from the hairpin deoxyoligonucleotides. In a preferred embodiment, the fluorescent DNA intercalator is either ethidium bromide or thiazole orange. If the set length is 4, then the plurality of hairpin deoxyoligonucleotides includes 136 hairpin deoxyoligonucleotides. If the set length is 5, then the plurality of hairpin deoxyoligonucleotides includes 512 isolated hairpin deoxyoligonucleotides.

Another apsect of the invention is directed to an article for use in a fluorescent intercalator displacement assay for determining DNA binding affinity and sequence selectivity of a test molecule. The article comprises one or more microtiter plates and a plurality of hairpin deoxyoligonucleotides. Each microtiter plate has an array of microtiter wells. Each hairpin deoxyoligonucleotide is physically separate from all other hairpin deoxyoligonucleotides within the plurality and is individually contained within and corresponds to one of the microtiter wells. Each hairpin deoxyoligonucleotide is present within its corresponding microtiter well with sufficient quantity for use the fluorescent intercalator displacement assay. Each hairpin deoxyoligonucleotide includes a single strand hairpin section and a double strand section. The single strand hairpin section consists of a non-variable sequence of unpaired deoxynucleotide bases. The double strand section includes a variable sequence of paired deoxynucleotide bases. The variable sequence has a set length of between 4 and 14 deoxynucleotide bases. The variable sequence is known or identifiable. The double strand section is attached to the single strand hairpin section. The plurality of hairpin deoxyoligonucleotides includes substantially all possible sequences within the set length of the variable sequence. The microtiter plates has a sufficient total number of microtiter wells for containing all of the plurality of hairpin deoxyoligonucleotides. In a further embodiment, the article further comprises a fluorescent DNA intercalator. The fluorescent DNA intercalator is intercalated into the hairpin deoxyoligonucleotides and is potentially displacable from said hairpin deoxyoligonucleotides by the test molecule. The fluorescent DNA intercalator is of a type that has altered fluorescent properties upon displacement from the hairpin deoxyoligonucleotides. In a preferred embodiment, the fluorescent DNA intercalator is either ethidium bromide or thiazole orange. If the set length is 4, then the plurality of hairpin deoxyoligonucleotides may include 136 hairpin deoxyoligonucleotides and the microtiter plates may have a total, all together, of at least 136 microtiter wells. If the set length is 5, then the plurality of hairpin deoxyoligonucleotides may include 512 hairpin deoxyoligonucleotides and the microtiter plates may have a total, all together, of at least 512 microtiter wells.

### BRIEF DESCRIPTION OF FIGURES

FIG. 1 shows the general procedure for the rapid DNA binding screen adaptable for determination of the sequence selectivity of a library of DNA binding agents.

FIG. 2 shows the hairpin structure of the nucleotides representing all possible combinations of five base pairs.

FIG. 3 shows the results of a screen of distamycin A against a library of DNA hairpin oligonucleotides.

FIG. 4 is a bar graph showing the influence of compound concentration of distamycin A on the percent fluorescence in the test with a given DNA sequence.

FIG. 5 is a bar graph showing the results of a test designed to find the concentration of DNA required to provide a robust assay reading.

FIG. 6 is a bar graph of a survey of concentration range for distamycin A versus a tight binding sequence (AATTT), and a modest binding sequence (AAACC).

FIG. 7 is two bar graphs which show the results of the ethidium bromide:DNA ratio and the influence of DNA concentration on percent fluorescence.

FIG. 8 is a table with the binding constants of distamycin A with particular short AT-rich sequences.

FIG. 9 is two plots. The first plot is of the linear region of the % fluorescence vs. distamycin A/DNA (base pairs). This graph can be extrapolated to estimate the number of distamycin molecules per length of DNA. Here the plot shows that about one molecule binds for every eight base pairs of DNA. The second plot shows the change in fluorescence versus the equivalents of EtBr. This graph yields the number of ethidium bromide molecules intercalated per hairpin. The linear regions are extrapolated to give a number of 3.3 molecules of ethidium bromide per hairpin.

FIG. 10 is a table showing the binding constants of ethidium bromide and thiazole orange.

FIG. 11 is a list of three equations used for a Scatchard analysis of titration binding curves.

FIG. 12 is two plots. The first plot shows the change in fluorescence versus equivalents of netropsin. This provides a titration curve from which the stoichiometry of binding can be derived. The second is a plot of DF/[Free Agent] versus DF which yields a linear portion of the Scatchard plot and provides −K as the slope of this portion of the curve.

FIG. 13 shows a screen of netropsin against a library of DNA hairpin deoxyoligo-nucleotides.

FIG. 14 is a table of netropsin binding constants.

FIG. 15 illustrates the screen of DAPI against a library of DNA hairpin oligonucleotides.

FIG. 16 is a table listing the binding constants for the four hairpin sequences first examined with distamycin A.

FIG. 17 is a plot of the titration of DAPI versus the hairpin containing 5′-ATTAA-3′ at 1.1 μM (8.8 μM bp) utilizing the inherent fluorescence of the DNA:DAPI complex.

FIG. 18 is the Scatchard plot for the titration of 5′-ATTAA-3′ utilizing the inherent fluorescence of the DNA:DAPI complex=−K.

FIG. 19 is a screen of Hoechst 33258 (2 μM concentration) against a library of DNA hairpin deoxyoligonucleotides.

FIG. 20 is a table of binding constants for Hoechst 33258 for four DNA sequences.

FIG. 21 is a screen of berenil against a library of DNA hairpin deoxyoligonucleotides.

FIG. 22 is a table of berenil binding constants with the four sequences that have been used to test the other compounds.

FIG. 23 is a graph of the titration curves for each of the five compounds tested with the hairpin containing 5′-AAAAA utilizing ethidium bromide displacement.

FIG. 24 is a table of statistical site frequency occurrence of the top 50sequences between the compounds.

FIG. 25 is a bar graph which illustrates the differing affinities for particular types of AT containing hairpins.

FIG. 26 is a bar graph that is an alternative way to examine the same data as FIG. 25.

FIG. 27 is two plots. The first one shows the change in fluorescence versus the number of equivalents of added netropsin. The netropsin is displacing thiazole orange from the DNA. The structure of thiazole orange is given above the plot. The linear portions of this plot are long and allow for a clean extrapolation to 1.03 equivalents of netropsin. The second plot is a Scatchard plot which gives the binding constant for thiazole orange to this particular hairpin DNA.

FIG. 28 is a table giving netropsin binding constants to four different hairpin DNA oligonucleotides. The two different constants were obtained by displacing thiazole orange and ethidium bromide from the hairpin DNA with netropsin.

### DETAILED DESCRIPTION

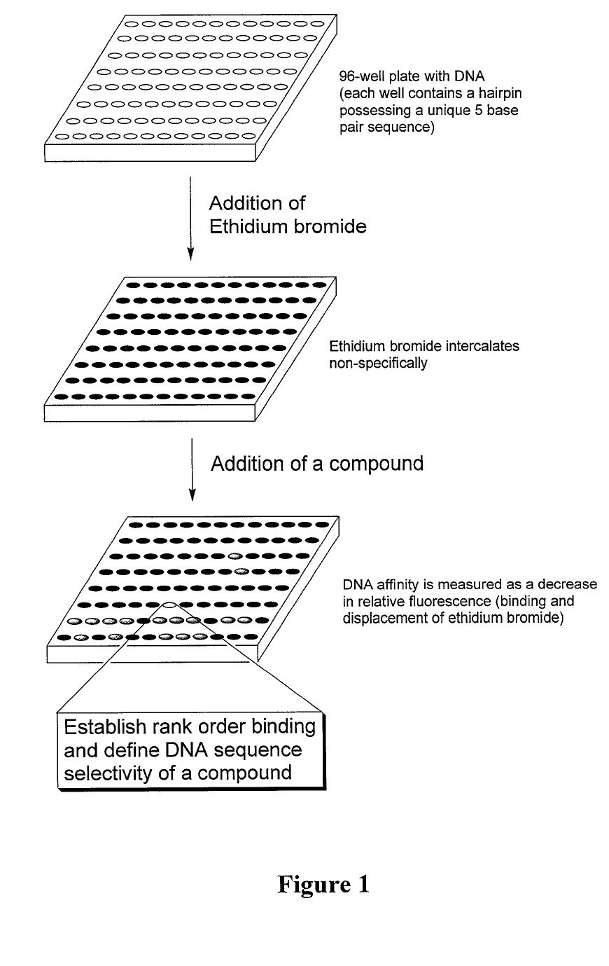

The nondestructive fluorescent intercalator displacement (FID) assay, described herein, utilizes the displacement of ethidium bromide or thiazole orange from hairpin deoxyoligonucleotides (FIG. 1). Hairpins containing all four (136 hairpins) or five base pair (512 hairpins) sequences individually displayed in 96-well plates are treated with the intercalator, yielding a fluorescence increase upon DNA binding (LePecq, J. -B.; Paoletti, C. J. Mol. Biol. 1967, 27, 87). Addition of a DNA binding compound results in a decrease in fluorescence due to displacement of the bound intercalator. The % fluorescence decrease is directly related to the extent of DNA binding providing relative binding affinities and a rank order binding to all possible five or four base pair (bp) sequences. The resulting profile defines the compound's sequence selectivity in high resolution and subsequent quantitative titration of any given hairpin sequence provides reliable binding constants (Jenkins, T. C. Optical Absorbance and Fluorescence Techniques for Measuring DNA-Drug Interactions, In Drug-DNA Interaction Protocols; Fox, K. R., Ed.; Methods in Molecular Biology; Humana Press: Totowa, N.J. 1997; Vol. 90, p 195; Morgan, A. R.; et al. Nucleic Acids Res. 1979, 7, 547.

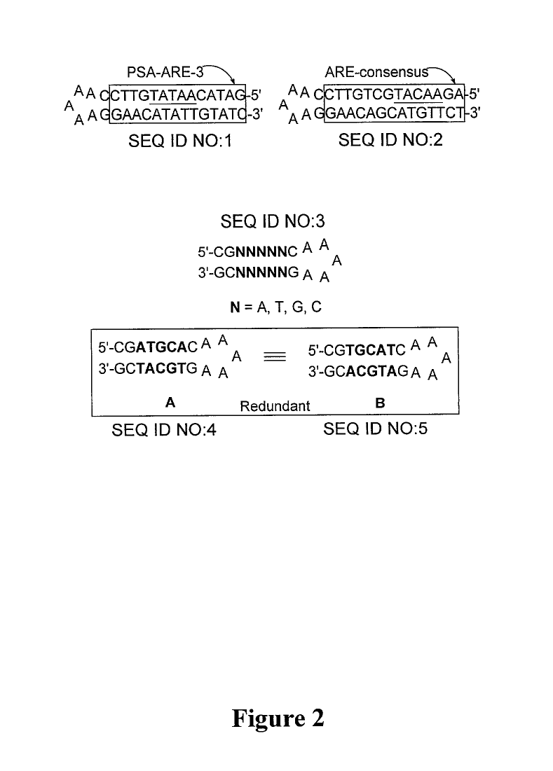

Distamycin A. Perhaps the most extensively studied DNA binding compound is distamycin A which binds to AT-rich sequences in the minor groove (Arcamone, F.; et al. Nature 1964, 203, 1064; Johnson, D. S.; Boger, D. L. DNA Binding Agents, In Comprehensive Supramolecular Chemistry; Vol. 4, J.-M. Lehn, Series Ed., Y. Murakami, Vol. Ed.; Pergamon: Oxford, England, 1996; pp 73-176 and references cited therein). Its DNA binding has been studied in detail by footprinting. However, even for distamycin A, a detailed study of its binding to all possible 5 bp sequences has not been described and its relative affinity for nonoptimal binding sites is not easily assessed using available techniques. Consequently, it represented an ideal case with which the technique could be first examined and parameters optimized. Thus, a survey of distamycin A binding to all five bp sequences was conducted with a library of 512 hairpin deoxyoligonucleotides each containing two of all possible five bp sequences in the general format 5′-CGNNNNNC-3′ with a 5-A loop (FIG. 2). Although there are 1024 possible sequences containing five bp, two complementary sequences are contained in each hairpin making, for example, the sequence 5′-ATGCA equivalent to the sequence 5′-TGCAT (FIG. 2). Such a library of binding sites is not limited to hairpins containing a variable five bp sequence. Smaller four bp sequences (5′-CGNNNNC-3′ with a 5-A loop, 136 hairpins) have also been used and larger sequences can also be employed.

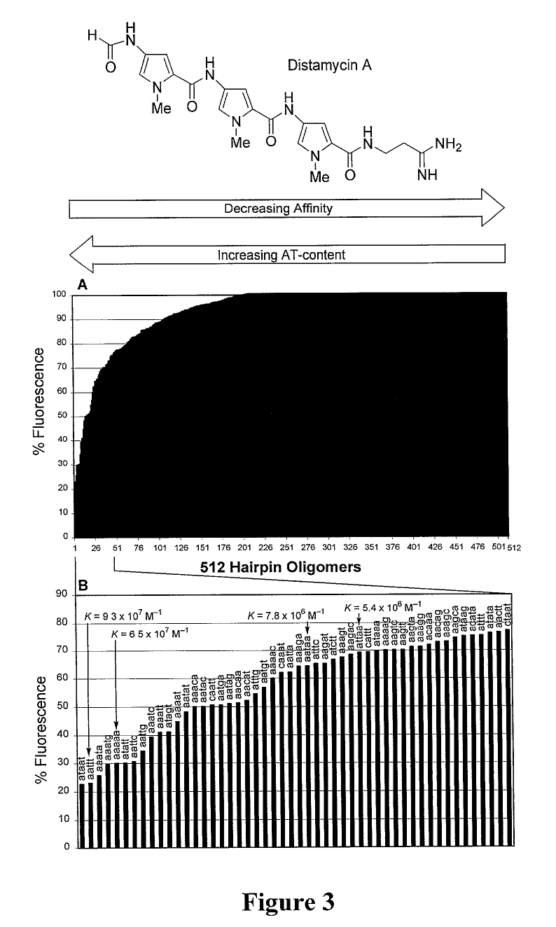

The results of screening the 512-member library of hairpins using distamycin A under a range of experimental parameters are summarized below, and those obtained under conditions recommended for use in a 96-well format are illustrated in FIG. 3. As expected, affinity increases with increasing AT content. All but two of the five bp AT sites were found in the top 45 sequences (1-45), essentially all four bp AT sites were observed in the top 151 sequences (4-151, 2 exceptions), and the three and two bp AT sites were observed in the range from 11 or 19 to 512 illustrating that distamycin A exhibits a clear selectivity for five and four bp AT sites over three and two bp AT sites. Thus, 14 of 16 five bp AT sites (88%), 15 of 32 four bp AT sites (47%), 10 of 80 three bp AT sites (13%), and a significant 11 of 176 two bp AT sites (6%) are found in the top 50 sequences. In contrast to the behavior of netropsin detailed later, 7 of the top 50 sequences (14%), but not the highest affinity sequences, contain a GC bp central to a five bp AT-rich site and this represents 7 of 16 such hairpin sites (44%). This distinct behavior has been noted in some footprinting studies and the data summarized in FIG. 3 provides a secure documentation of this observation and its relative importance.

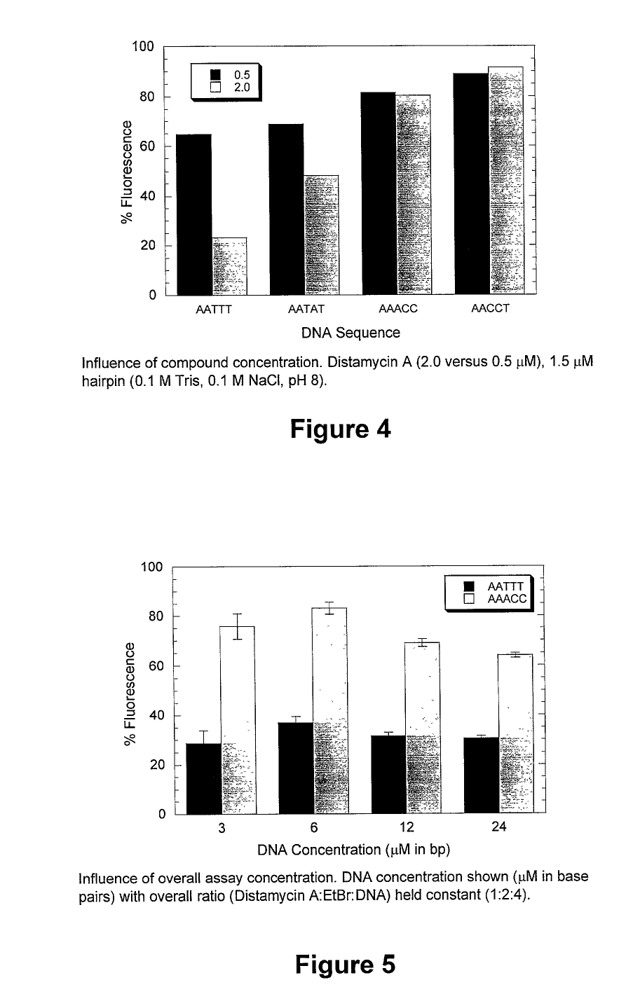

Compound Concentration Dependence. Among the first parameters examined was the impact of the compound concentration. The assays were conducted at a single concentration of the hairpin (1.5 μM, 12 μM in bp) and under buffer conditions (0.1 M Tris, 0.1 M NaCl, pH 8) that represent relatively high salt concentrations which approximate physiologically relevant conditions and minimize simple electrostatic binding to DNA. This minimizes the lower affinity binding of ethidium bromide to the phosphate backbone that could complicate the assay. Optimally, the measured % fluorescence decrease should be sufficiently intense to provide a robust signal, and the dynamic range sufficiently large that comparisons of compounds with different affinities could be made at a single concentration. For assays conducted with 1.5 μM hairpin, 1 or 2 μM compound concentrations met these criterion (FIG. 4). Use of lower compound concentrations resulted in less discrimination between sequences (0.5 vs 2.0 μM) and higher compound concentrations similarly led to less distinction, especially among the higher affinity sequences. This use of a near 1:1 ratio of compound to DNA provided the desired robust intensity of measured % fluorescence decrease suitable for a high throughput assay across a range of compounds with varied affinities. Typically, the most useful concentration of compound proved to be either 1 or 2 μM, bracketing the concentration of DNA enlisted in the assay (1.5 μM).

Assay Concentration. In the initial studies, the concentration of DNA required to provide a robust assay reading was examined. The intention was to minimize the amount of DNA and reagents needed in a standard 96-well format (Costar black opaque, 100 μL assay volume) while maintaining a reliable measurement. The hairpin concentration for a high affinity (AATTT) and lower affinity sequence (AAACC) was varied from 0.375-3.0 μM (3-24 μM bp) maintaining a 1:2 ethidium bromide:bp ratio and a constant ratio of distamycin A:hairpin (FIG. 5). To a first approximation, the % fluorescence decrease upon distamycin A binding should not vary with this change in concentration. Concentrations of 1.5 μM hairpin (12 μM bp) or higher in a 100 μL volume maintained an acceptably constant reading to be reliable for both hairpins whereas those below 1.5 μM suffered variations too large to be considered useful (FIG. 5). Consequently, a hairpin concentration of 1.5 μM was adopted herein.

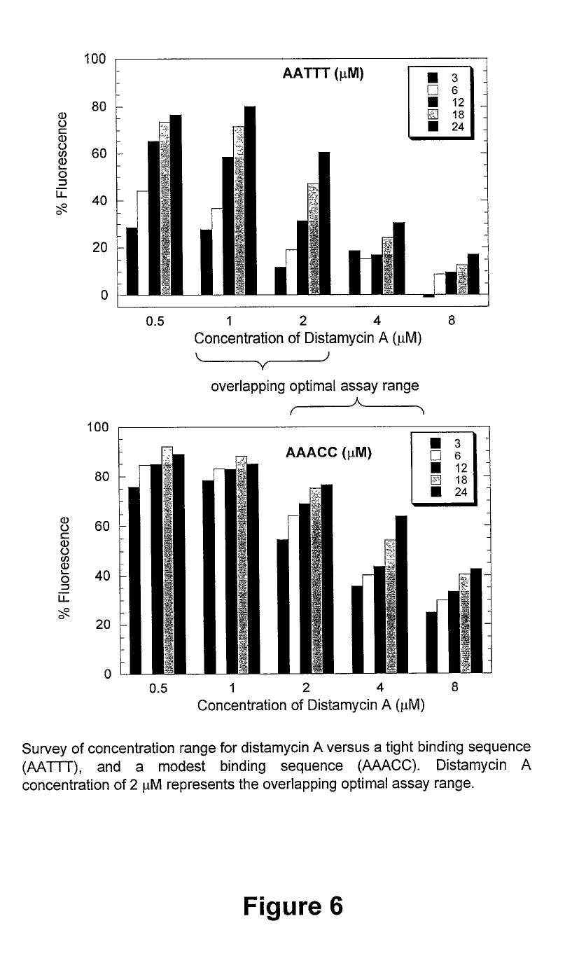

Assay Concentration Range. Several studies were conducted to establish an optimal assay concentration and single compound concentration that could be used in a high throughput assay. This led to the identification of conditions that would discriminate among high and lower affinity sites for tight or even modest binding agents. One representative study is illustrated in FIG. 6 with distamycin A and two sequences (AATTT and AAACC). With the high affinity AATTT sequence, concentrations as low as 0.5 μM and optimally 1 or 2 μM distamycin A performed well across the range of hairpin concentrations (3-24 μM bp). With the lower affinity AAACC sequence, concentrations of 2-8 μM (2-4 μM optimally) distamycin A performed well across the range of hairpin concentrations whereas the lower concentrations of 0.5-1 μM were not useful regardless of the hairpin concentration. The optimal conditions that accommodate both the high (AATTT) and modest (AAACC) affinity sites is the 2 μM distamycin A concentration and the midrange 12 μM bp hairpin (1.5 μM) concentration, the conditions adopted for the high throughput assay.

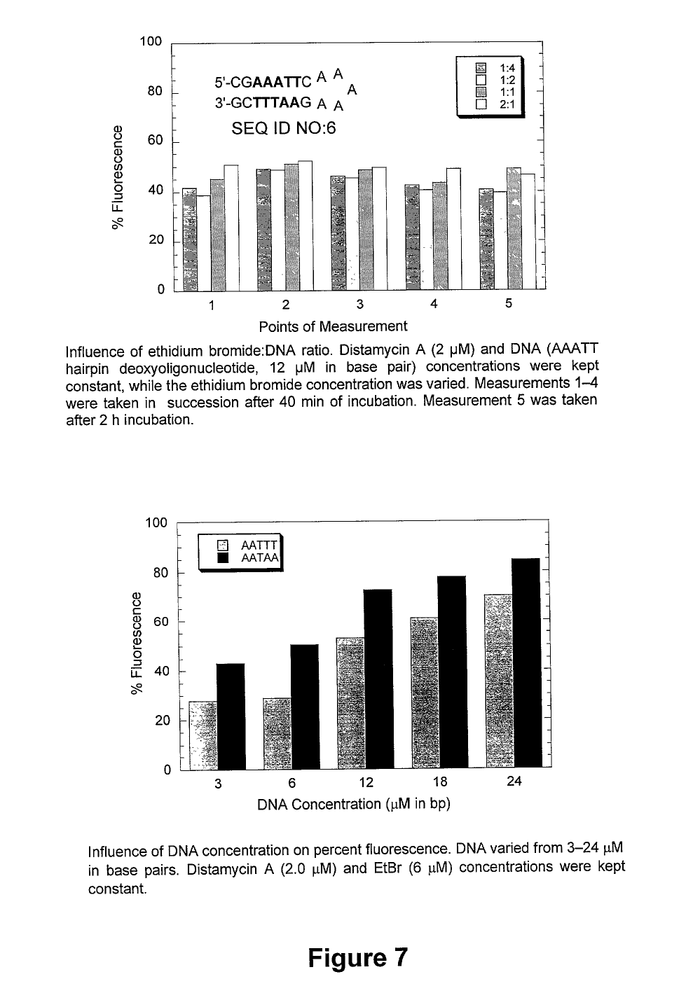

Ratio of Ethidium Bromide to DNA. One special concern was the sensitivity of the method to the amount of ethidium bromide employed. The method was expected to perform best at a 1:2 ethidium bromide:base pair ratio (EtBr:bp) where all intercalation sites are occupied. However, it was not clear what impact deviations from this ratio would have. Under conditions where the salt concentration is modestly high and approximating that which is physiologically relevant, little impact was observed when this ratio was varied over a range of 1:4 to 2:1 EtBr:bp. Consistent with the conditions adopted in the assay, the greatest % fluorescence decrease was observed at the optimal 1:2 EtBr:bp ratio (FIG. 7). The slightly lower decrease in % fluorescence for the 1:4 EtBr:bp ratio underestimates the compound binding since not all available intercalation sites are occupied and compound binding can occur at sites where less or no intercalator is displaced. The % fluorescence decrease also diminished slightly as the ethidium bromide concentration was raised above the optimal 1:2 EtBr:bp ratio presumably due to enhanced background fluorescence. However, the method proved surprisingly independent of this variable. Through repeated measurements on the same samples, the % fluorescence decrease variation was typically ±8% in this 1:4 to 2:1 EtBr:bp ratio range. Consequently, use of the 1:2ratio is recommended. Variations may be expected if this ratio is not consistently maintained. However, this ratio does not appear to be critical and minor experimental variations are unlikely to have a significant impact.

Measurement Time, Number, and Assay Variability. The assay variability and the impact of repeated measurements using a 96-well format assay and a fluorescent plate reader were examined. Black Costar 96-well plates with a flat bottom and a Spectra Max Gemini fluorescent plate reader from Molecular Devices that records and averages 30 fluorescent readings per well were employed. With the exception of the initial reading, the % fluorescence decrease diminished slightly with time/reading indicating some level of photobleaching with each reading (FIG. 7). Importantly, no alterations in the relative % fluorescence decrease readings were observed within each measurement grouping indicating that the qualitative rankings are not affected. However, the absolute readings show considerable variation which are as large as ±10% fluorescence decrease (1:2 EtBr:bp ratio) to as small as ±5% (2:1 ratio). This suggests that variation in the assay may be expected from run to run, but that the ranking within a given run may be considered reproducibly accurate. For more quantitative comparisons including titrations to establish binding constants or for careful comparison of side-by-side sequences in a ranked profile, the use of 96-well format and plate readers is not recommended. Instead, the use of a 3 mL cuvette for the compound titrations is recommended.

Hairpin Deoxyoligonucleotide Quality. The variable that is critical to the success of the assay, and most likely to be responsible for avoidable errors, is the quality of the hairpins. In addition to the obvious concern of its constitution and purity, its concentration is critical and must be determined by measuring the UV absorption (260 nm) of the denatured, single-stranded DNA at 80-95° C. (0.01 M Na₂HPO₄/H₃PO₄, 0.1 M NaCl, pH 7). Given that each 21-mer the library exists as a hairpin duplex at 25° C., where this construct represents a combination of double and single-stranded DNA, calculations of the single strand oligo concentration based on UV absorption measurements at 25° C. underestimate the concentration by approximately 25% (range 18-32%) (This range was determined using the absorbence value at 25° C. to calculate DNA concentration and compared with the same calculation using the absorbence determined at 80-95° C. This procedure was conducted on a representative array of hairpins with varying GC content to provide the range of error.). Consequently, the concentration of each hairpin in this library was determined by measuring the UV absorbance at 90° C. In the course of making these measurements, all hairpins in the library displayed melting curves which indicated they adopt stable duplex structures at 25° C. Unlike the use of simple deoxyoligonucleotides which may suffer from sequence-dependent single-strand/duplex equilibria under the conditions of this assay, the hairpins form stable duplex structures. An indication of the errors that might be introduced if the hairpin concentration is not addressed accurately is illustrated in FIG. 7. Measurements conducted with hairpin concentrations lower than expected (e.g., 3 and 6 vs 12 μM bp) yield much larger than expected % fluorescence decreases, falsely indicating higher affinity binding. Similarly, measurements conducted with hairpin concentrations higher than expected (18 and 24 vs 12 μM) result in lower than expected % fluorescence decreases and underestimate binding affinity. These alterations in the measured values are significant and indicate that this variable alone is the one most important to control. However, it is not the absolute concentration of the hairpin that is important, rather it is important that all hairpins are used at the same concentration for determination of a ranked binding profile.

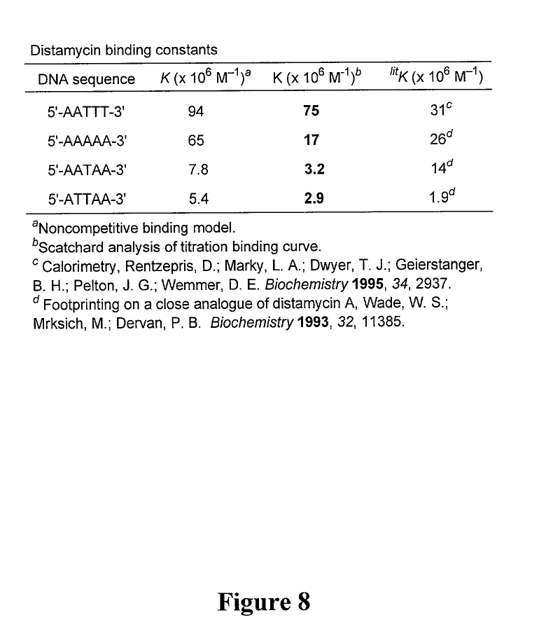

Binding Constants. Although distamycin A has been studied in detail, few binding constants for short AT-rich sequences have been established. The comparison of those disclosed show the relative trend of 5′-AATTT>AAAAA>AATAA>ATTAA (FIG. 8) (Rentzeperis, D.; et al. Biochemistry 1995, 34, 2937; Wade, W. S.; et al. Biochemistry 1993, 32, 11385). The ethidium bromide displacement assay revealed the same general trend and a quantitative titration with the hairpins containing these sequences afforded binding constants that are not only consistent with the relative trend (FIG. 3), but also within a factor of 2-4 of the binding constants determined through calorimetry or DNase I footprinting (FIG. 8). Given that the DNAs upon which the measurements were made are different, that the pH and buffer conditions are not identical, and that entries 2-4 in FIG. 8 were derived with a close analogue of distamycin A, all which may contribute to small discrepancies in the binding constant values, the method appears to be accurate at providing absolute binding constants. Three approaches to establishing the binding constants from a quantitative titration based on the displacement of ethidium bromide were compared. Two of these, involving the use of a noncompetitive (Morgan, A. R.; et al. Nucleic Acids Res. 1979, 7, 547) or competitive (Jenkins, T. C. Optical Absorbance and Fluorescence Techniques for Measuring DNA-Drug Interactions, In Drug-DNA Interaction Protocols; Fox, K. R., Ed.; Methods in Molecular Biology; Humana Press: Totowa, N.J. 1997; Vol. 90, p 195) binding model, were detailed and discussed in an earlier publication (Boger, D. L.; et al. J. Am. Chem. Soc. 2000, 122, 6382; Boger, D. L.; et al. Bioorg. Med. Chem. 2000, 8, 2049; Boger, D. L.; Lee, J. K. J. Org. Chem. 2000, 65, 5996; Boger, D. L.; et al. J. Org. Chem. submitted). In the intervening time, it has been found that a third method involving a Scatchard (Scatchard, G. Ann. NY Acad. Sci. 1949, 51, 660; Perkins, H. R. Biochem. J. 1969, 111, 195; Schmitz, H.-U.; Hübner, W. Biophys. Chem. 1993, 48, 61) or curve fitting analysis of the titration curves (Brown, K. A.; et al. J. Am. Chem. Soc. 1993, 115, 7072; Satz, A. L.; Bruice, T. C. Bioorg. Med. Chem. 2000, 8, 1871; Satz, A. L.; Buice, T. C. J. Am. Chem. Soc. 2001, 123, 2469. For additional discussions of multiple equilibria or multiple binding modes and the limitations of a Scatchard analysis, see: Norby, J. G.; et al. Anal. Biochem. 1980, 102, 318; Feldman, H. A. Anal. Biochem. 1972, 48, 317; Deranleau, D. A. J. Am. Chem. Soc. 1969, 91, 4050; Glasel, J. A.; et al. J. Biol. Chem. 1976, 251, 2929) provides more satisfactory results. The noncompetitive binding model is experimentally difficult to implement (r-value determination), but can provide reliable constants, whereas the often used competitive binding model fails to provide reliable estimates of K (A detailed discussion of the noncompetitive and competitive binding models is provided in Supporting Information along with a discussion of limitations to their use.).

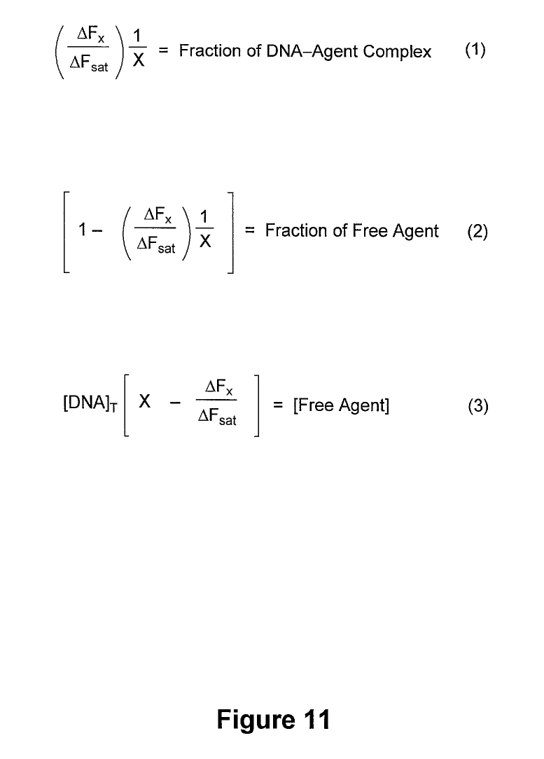

This third method of establishing binding constants, which has been used extensively by Bruice (Brown, K. A.; et al. J. Am. Chem. Soc. 1993, 115, 7072; Satz, A. L.; Bruice, T. C. Bioorg. Med. Chem. 2000, 8, 1871; Satz, A. L.; Buice, T. C. J. Am. Chem. Soc. 2001, 123, 2469. For additional discussions of multiple equilibria or multiple binding modes and the limitations of a Scatchard analysis, see: Norby, J. G.; et al. Anal. Biochem. 1980, 102, 318; Feldman, H. A. Anal. Biochem. 1972, 48, 317; Deranleau, D. A. J. Am. Chem. Soc. 1969, 91, 4050; Glasel, J. A.; et al. J. Biol. Chem. 1976, 251, 2929), provides the most reliable means of determining K and the stoichiometry of binding, and is easily extended to analyzing higher order 2:1 or 3:1 complexes. In Bruice's work, this has entailed either the competitive displacement of Hoechst 33258 and a measurement of the resulting fluorescence decrease or a direct measurement of a fluorescent change in the DNA binding compound itself. It is disclosed that the former works equally well in the assays measuring the displacement of prebound ethidium bromide or thiazole orange. For cases in which the binding stoichiometries are 1:1, binding constants may be established by Scatchard analysis (Scatchard, G. Ann. NY Acad. Sci. 1949, 51, 660; Perkins, H. R. Biochem. J. 1969, 111, 195; Schmitz, H.-U.; Hübner, W. Biophys. Chem. 1993, 48, 61) of the titration binding curves. Thus, equations (1)-(3) (FIG. 11) are used to establish the free agent concentration employed to generate a Scatchard plot from which binding constants may be determined. In these equations, [Free Agent]=concentration of free agent, [DNA]_{T}=total concentration of DNA, X=molar equiv of agent versus DNA, ΔFₓ=change in fluorescence, ΔFₛₐₜ=change in fluorescence at the point where DNA is saturated with ligand.

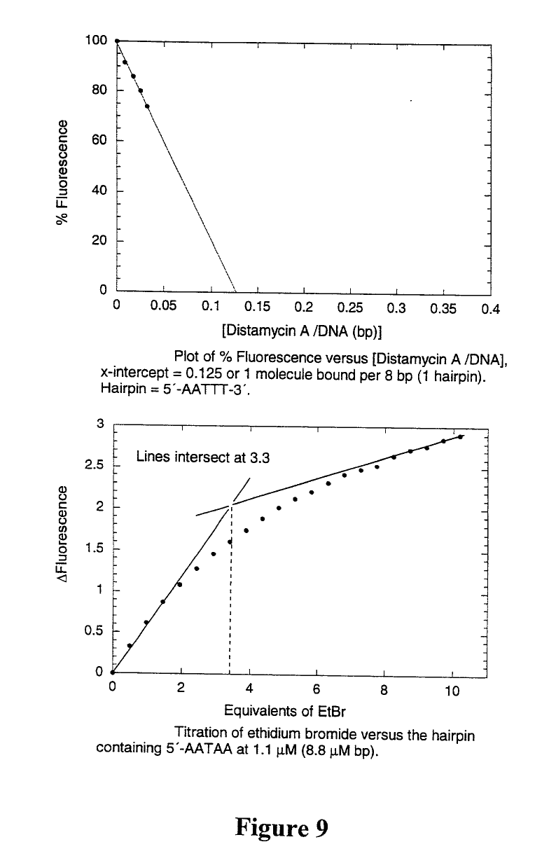

A plot of the change in fluorescence versus equiv of compound provides a titration curve from which the stoichiometry may be determined (FIG. 9). The mathematical intersection of the pre and post-saturation portions of the curve provides ΔFₛₐₜ and allows for the determination of [Free Agent]. The plot of ΔF/[Free Agent] versus ΔF yields a linear portion of the Scatchard plot and provides −K as the slope of this portion of the curve (FIG. 9).

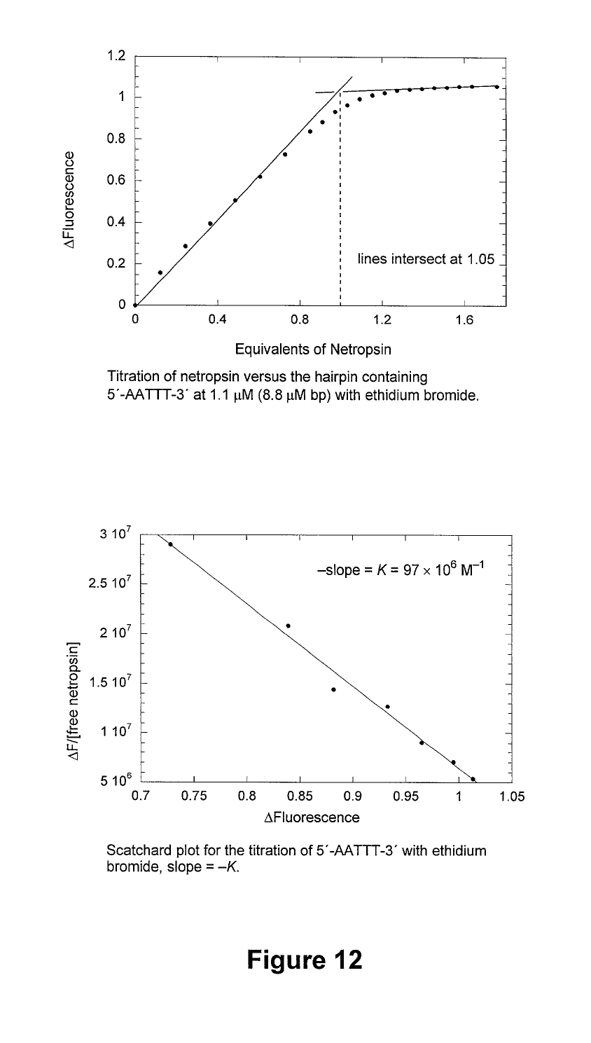

Bruice has emphasized that such reciprocal plotting techniques are restrictive and in more complex systems (e.g., 2:1 or 3:1 binding) can lead to errors in interpreting the data (Brown, K. A.; et al. J. Am. Chem. Soc. 1993, 115, 7072; Satz, A. L.; Bruice, T. C. Bioorg. Med. Chem. 2000, 8, 1871; Satz, A. L.; Buice, T. C. J. Am. Chem. Soc. 2001, 123, 2469. For additional discussions of multiple equilibria or multiple binding modes and the limitations of a Scatchard analysis, see: Norby, J. G.; et al. Anal. Biochem. 1980, 102, 318; Feldman, H. A. Anal. Biochem. 1972, 48, 317; Deranleau, D. A. J. Am. Chem. Soc. 1969, 91, 4050; Glasel, J. A.; et al. J. Biol. Chem. 1976, 251, 2929). In such cases, iterative curve fitting of the experimental points provides reliable results and can dissect multiple equilibria. For the analysis of 1:1 binding as illustrated for netropsin in FIG. 12, this is unnecessary. Both the Scatchard and the curve fitting analysis provide nearly identical values for K. Given the relative ease of the use of this technique and the reliability with which visual inspection of the plotted data can confirm the assumption of 1:1 binding, the use of the Scatchard analysis of the titration binding curves for determining K is recommended.

In two of the examples which follow, binding constants could be assessed by this indirect technique involving the displacement of ethidium bromide or directly by measuring the fluorescent increase of the DNA binding compound itself, i.e., in the absence of a competitive binding agent. For the compounds examined to date, the direct and indirect methods provided comparable results that do not appear to be affected by the sequence dependent affinity of ethidium bromide, or the stoichiometry of binding displacement.

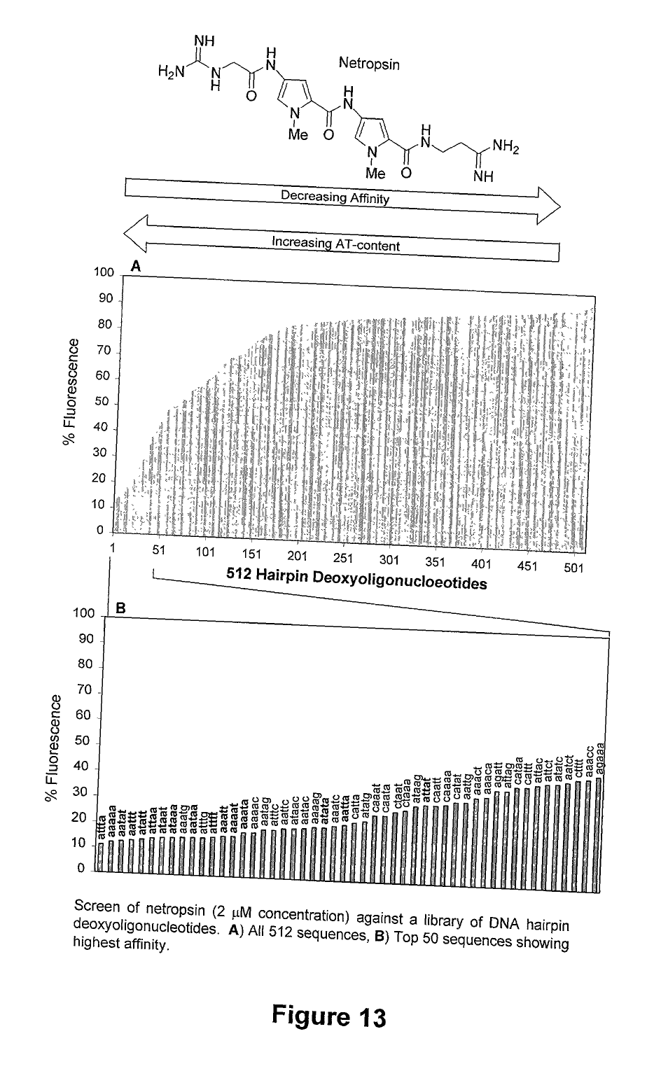

Netropsin. Early biophysical studies of Wells and Zimmer (Finlay, A. C.; et al. J. Am. Chem. Soc. 1951, 73, 341; Wartell, R. M.; et al. J. Biol. Chem. 1974, 249, 6719; Wells, R. D.; et al. Prog. Nucleic Acids Res. Molec. Biol. 1980, 24, 167; Zimmer, Ch.; et al. J. Mol. Biol. 1971, 58, 329; Zimmer, Ch. Prog. Nucleic Acid Res. Molec. Biol. 1975, 15, 285; Zimmer, Ch.; Wähnert, U. Prog. Biophys. Molec. Biol. 1986, 47, 31) and more recent footprinting (Harshman, K. D.; Dervan, P. B. Nucleic Acids Res. 1985, 13, 4825; Van Dyke, M. W.; et al. Proc. Natl. Acad. Sci. USA 1982, 79, 5470), NMR (Patel, D. J.; Canuel, L. L. Proc. Natl. Acad. Sci. USA 1977, 74, 5207; Patel, D. J. Eur. J. Biochem. 1979, 99, 369; Patel, D. J. Proc. Natl. Acad. Sci. USA 1982, 79, 6424; Patel, D. J.; Shapiro, L. Biochimie 1985, 67, 887; Patel, D. J.; Shapiro, L. J. Biol. Chem. 1986, 261, 1230; Patel, D. J.; Shapiro, L. Biopolymers 1986, 25, 707; Ashcroft, J.; et al. Biopolymers 1991, 31, 45; Sarma, M. H.; et al. J. Biomol. Struct. Dyn. 1985, 2, 1085; Gupta, G.; et al. J. Biomol. Struct. Dyn. 1984, 1, 1457), X-ray (Dickerson, R. E.; Kopka, M. L. J. Biomolec. Struct. Dyn. 1985, 2, 423; Kopka, M. L.; et al. J. Mol. Biol. 1985, 183, 553; Kopka, M. L.; et al. Proc. Natl. Acad. Sci. USA 1985, 82, 1376; Kopka, M. L.; et al. In Structure & Motion: Membranes, Nucleic Acids & Proteins; Clementi, E., Corongiu, G., Sarma, M. H., Eds.; Adenine Press: New York, 1985, 461. Nunn, C. M.; et al. Biochemistry 1997, 36, 4792; Tabernero, L.; et al. Biochemistry 1993, 32, 8403), and calorimetry studies (Breslauer, K. J.; et al. In Structure & Expression, Vol. 2: DNA and Its Drug Complexes; Sarma, R. H., Sarma, M. H., Eds.; Adenine Press: Schenectady, N.Y., 1988, 273) have characterized netropsin's minor groove AT binding selectivity. Its affinity matches or exceeds that of distamycin and it has been characterized by a smaller four versus five bp AT selectivity. Netropsin behaved exceptionally well in the assay, so much so that it is recommended for use in its validation (FIG. 13). Consistent with expectations, the affinity increases with increasing AT content. Even more smoothly than distamycin A, netropsin exhibited a 5>4>3 bp AT selectivity. The top 8 sequences and 13 of the top 15 sequences were five versus four bp AT sites. All five bp AT sequences were found in the top 33 sequences. Thus, 16/16 five bp AT sites (100%), 27/32 four bp AT sites (84%), but only 7/80 three bp AT sites were found in the top 50 sequences (9%). Unlike distamycin A, no AT-rich sequence interrupted by a central GC bp appears in the top 50 sequences and no three bp AT sites appear before haripin 38. As such, the selectivity for a four or five bp AT site is more strictly adhered to by netropsin than distamycin.

Netropsin was also examined at 1.0 and 0.5 μM concentrations. Consistent with expectations, the assay performed similarly at 1.0 μM netropsin, but exhibited greater variation in the sequence ordering at 0.5 μM due to greater intrinsic error in the measurements. In this regard, it is important to recognize that a minor reordering of the sequences is expected from run to run, or upon altering the agent concentration since the difference in the % fluorescence decrease measured for most deoxyoligonucleotides adjacent to one another in the binding profiles is within the experimental variation of the assay. For example, ATTTA was identified as sequence no. 1 and 3 at 2.0 and 1.0 μM netropsin, respectively.

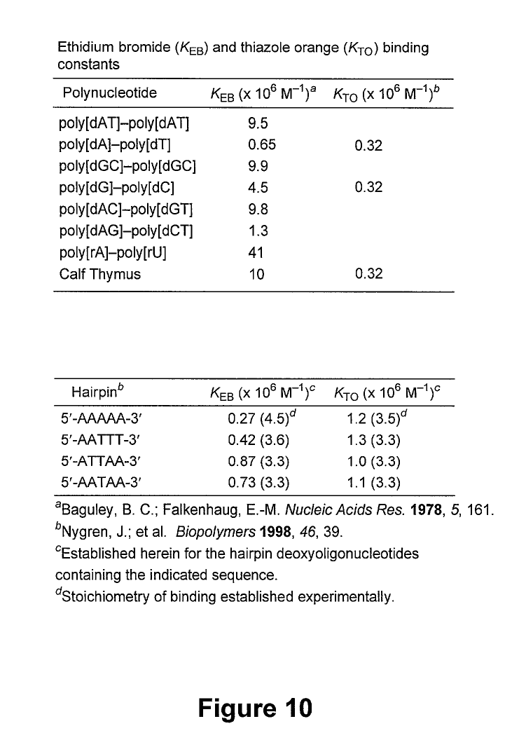

The netropsin binding constants were established for several hairpin sequences by quantitative titration using the four sequences enlisted for distamycin A (FIG. 10). The binding clearly reflects a 1:1 stoichiometry (see FIG. 12) and the Scatchard analysis of the titration binding curves was well behaved. In general, the affinities for netropsin were approximately 5-25 fold greater than distamycin A.

DAPI. 4′,6-Diamidine-2-phenylindole.2HCl (DAPI) (Dann, O.; et al. Justus Liebigs Ann. Chem. 1971, 749, 68), is widely used as a fluorescent dye for DNA and chromosomes (Kapuscinski, J.; Skoczylas, B. Nucleic Acids Res. 1978, 5, 3775; Russell, W. C.; et al. Nature 1975, 253, 461; Williamson, D. H.; Fennell, D. J. Methods Enzymol. 1979, 56, 728). Extensive studies have shown that DAPI binds selectively to AT-rich sites in the minor groove (Kapuscinski, J.; Szer, W. Nucleic Acids Res. 1979, 6, 3519; Manzini, G.; et al. Nucleic Acids Res. 1983, 11, 8861; Kubista, M.; et al. Biochemistry 1987, 26, 4545; Portugal, J.; Waring, M. Biochim. Biophys. Acta 1988, 949, 158). Early studies (Kania, J.; Fanning, T. G. Eur. J. Biochem. 1976, 67, 367; Chandra, P.; Mildner, B. Cell Mol. Biol. 1979, 25, 137) postulated an intercalating binding mode and Wilson later demonstrated that DAPI can interact with GC-rich regions by intercalation albeit with binding that is 100-1000 fold weaker than its AT-rich minor groove binding (Wilson, W. D.; et al. J. Am. Chem. Soc. 1989, 111, 5008). Footprinting experiments revealed that DAPI requires three or more consecutive AT bp to bind in the minor groove (Jeppesen, C.; Nielsen, P. E. Eur. J. Biochem. 1989, 182, 437), X-ray (Larsen, T. A.; et al. J. Biomol. Struct. Dyn. 1989, 7, 477) and NMR (Trotta, E.; et al. J. Biol. Chem. 1993, 268, 3944; Loontiens, F. G.; et al. Biochemistry 1991, 30, 182) studies have confimed a 1:1 binding stoichiometry, and the latter have also established a preference for a 4>3 >2 bp AT binding site (Loontiens, F. G.; et al. Biochemistry 1991, 30, 182).

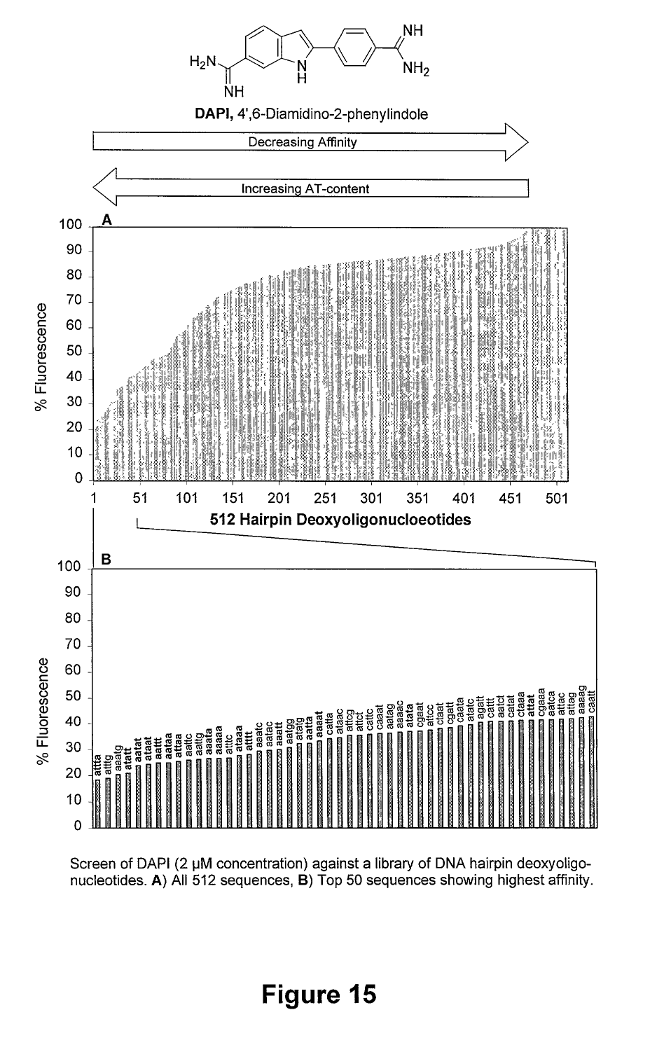

DAPI was examined against the set of 512 hairpins at a 2 μM concentration and exhibited a high selectivity for a binding site containing at least three AT bp (FIG. 15). All but one of the five bp AT sites were in the top 34 sequences (1-34), essentially all four bp AT sites were observed in the top 90 sequences (2-90, 4 exceptions), the three bp AT sites were observed in the range of 17-185 (2 exceptions), and the two bp AT sites were observed only in the higher range of >70. Thus, 16 of 16 five bp AT sites (100%), 23 of 32 four bp AT sites (72%), and 11 of 80 three bp AT sites (14%) are found in the top 50sequences. Like netropsin, but unlike distamycin A, none of the top 50 sequences contain a GC bp central to a five bp AT-rich site. The weaker binding observed with non AT-rich hairpins (>150-512, % fluorescence decrease <20%), which was not observed with distamycin A, most likely represents the weaker DAPI intercalation first documented by Wilson. The merged bar graph of the 512 sequences exhibits a slope that is shallower and an area under the curve that is smaller than those of distamycin A, indicating that DAPI binds effectively to more sequences and exhibits less sequence selectivity than distamycin A.

One key element that should be emphasized is that the fluorescence enhancement characteristic of the DAPI binding does not interfere with the measurement of the fluorescent decrease derived from the ethidium bromide displacement. This is a consequence of the ethidium fluorescence measurement made with excitation at 545 nm and an emission at 595 nm that is unaffected by the DAPI absorption (372 nm) and emission (454 nm). Thus, the generality of the method extends even to compounds that themselves exhibit fluorescent properties. The binding of DAPI to the library of hairpins could also be conducted such that its own intrinsic fluorescent enhancement was monitored and relative binding established by a % fluorescence increase.

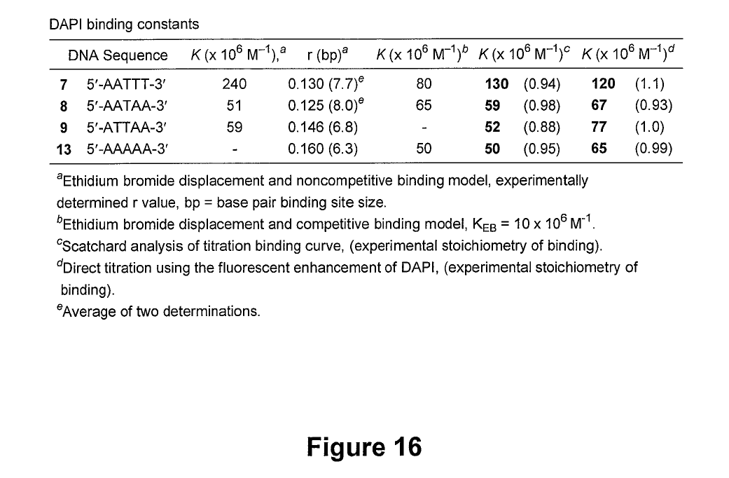

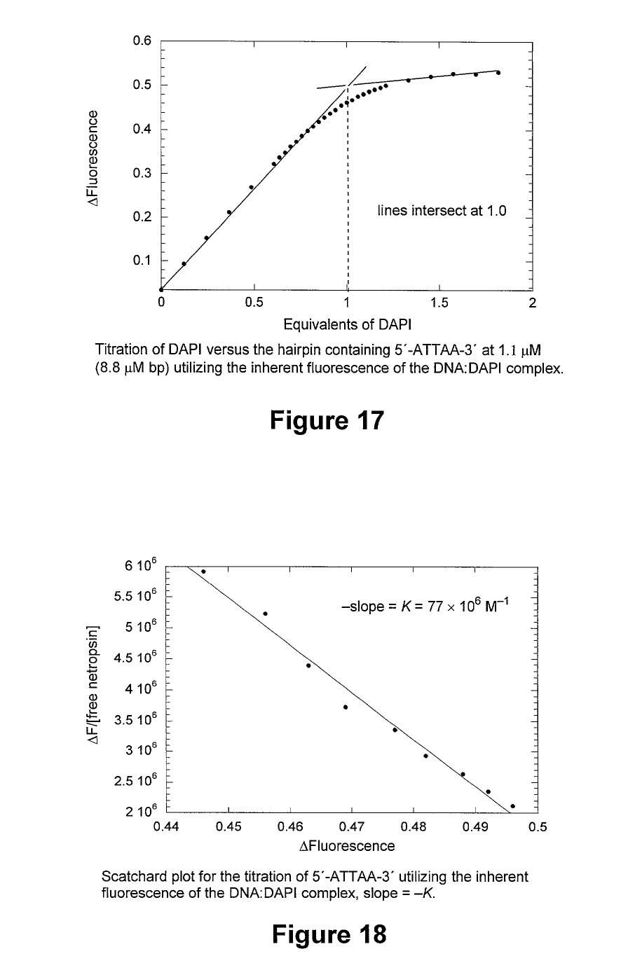

The binding constants for DAPI were determined for the four hairpins first examined with distamycin A (FIG. 16). Although no binding constants established by other methods were found these same sequences, closely related sequences have been studied by footprinting, or direct fluorescence titration (Loontiens, F. G.; et al. Biochemistry 1991, 30, 182). In addition, the characteristic fluorescence enhancement of DAPI upon DNA binding could be monitored in a direct titration in the absence of added ethidium bromide and used to independently establish binding constants and the 1:1 binding stoichiometry. The values obtained by this direct titration match closely, but are slightly higher than those obtained in the ethidium bromide displacement titration validating the accuracy of this latter technique (FIG. 16). Upon direct titration of the four hairpins, the resulting plot of fluorescence versus the number of equivalents of DAPI illustrated saturated binding upon addition of one equivalent of DAPI (FIGS. 17 and 18).

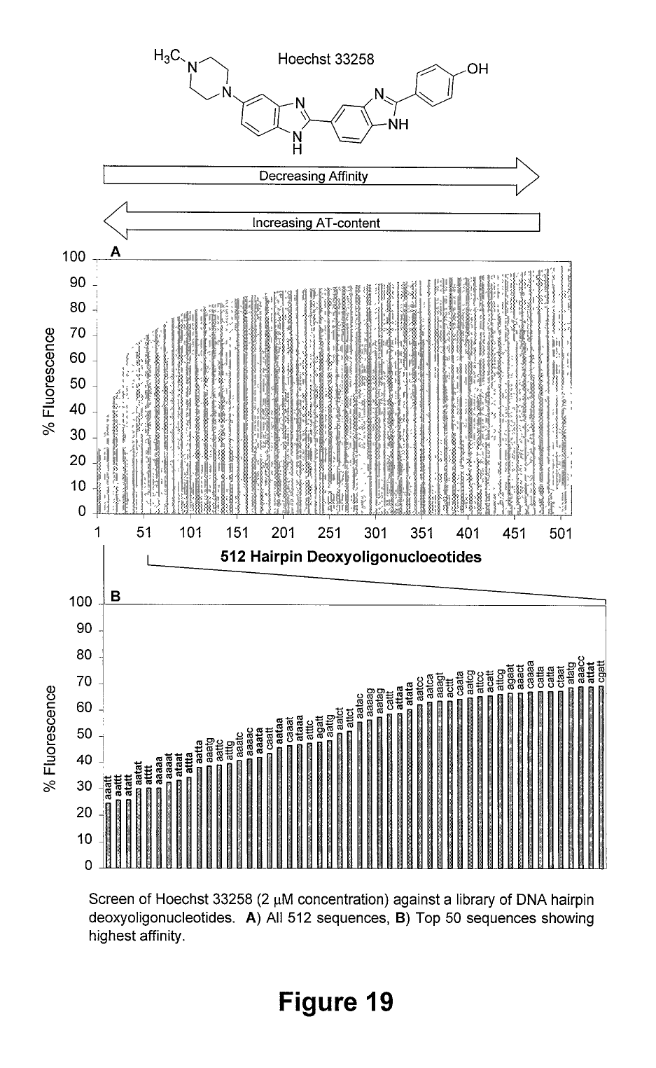

Hoechst 33258. An additional fluorescent DNA stain, Hoechst 33258 (Bontemps, J.; et al. Nucleic Acids Res. 1975, 2, 971), has been shown to selectively bind minor groove four and five bp AT sites (Mikhailov, M. V.; et al. Mol. Biol. (Engl. Trans.) 1981, 15, 541; Martin, R. F.; Holmes, N. Nature 1983, 302, 452). Both NMR (Searle, M. S.; Embrey, K. J. Nucleic Acids Res. 1990, 18, 3753; Parkinson, J. A.; et al. Biochemistry 1990, 29, 10181; Fede, A.; et al. Biochemistry 1991, 30, 11377; Fede, A.; et al. Structure 1993, 1, 177; Embrey, K. J.; et al. Eur. J. Biochem. 1993, 211, 437; Parkinson, J. A.; et al. Biochemistry 1994, 33, 8442; Gavathiotis, E.; et al. Nucleic Acids Res. 2000, 28, 728) and X-ray studies (Pjura, P. E.; et al. J. Mol. Biol. 1987, 197, 257; de C. T. Carrondo, M. A. A. F.; et al. Biochemistry 1989, 28, 7849; Teng, M.-k.; et al. Nucleic Acids Res. 1988, 16, 2671; Sriram, M.; et al. EMBO J. 1992, 11, 225; Quintana, J. R.; et al. Biochemistry 1991, 30, 10294) have confirmed the AT-rich minor groove binding and some of the latter X-ray studies, but not the former NMR studies, suggest an interaction of the N-methylpiperazine with a terminal GC bp. X-ray, NMR, and calorimetric studies revealed a 1:1 binding stoichiometry (Haq, I.; et al. J. Mol. Biol. 1997, 271, 244). Footprinting studies (Kapuscinski, J.; Szer, W. Nucleic Acids Res. 1979, 6, 3519; Manzini, G.; et al. Nucleic Acids Res. 1983, 11, 8861; Kubista, M.; et al. Biochemistry 1987, 26, 4545; Portugal, J.; Waring, M. Biochim. Biophys. Acta 1988, 949, 158; Routier, S.; et al. Nucleic Acids Res. 1999, 27, 4160) have further illustrated that Hoechst 33258 protects a five bp region within AT-rich sequences. Notably, these studies also corroborate the X-ray observation of a tolerance for AT-rich sequences containing isolated GC base pairs. Binding to GC-rich sequences has also been detected by a process that does not involve external or groove binding and has been attributed to a weaker intercalation binding (Bailly, C.; et al. Nucleic Acids Res. 1993, 21, 3705). Consistent with these observations, Hoechst 33258 exhibited AT-rich binding when assayed against the library of 512 hairpins (FIG. 19). Its selectivity lies somewhere between that of DAPI and distamycin A and this is clear from a comparison of the merged bar graphs of the 512 hairpin binding results. All three exhibit comparable binding to the best sequences, but the rise of the graph or slope of the plot lies between those of DAPI and distamycin A and the area under the curve indicates that the selectivity follows the order distamycin A>Hoechst 33258>DAPI. Thus, 16 of 16 five bp AT sites (100%) and 19 of 32 four bp AT sites (59%) are found in the top 50 sequences. Although the top 20 sequences all contain five or four bp AT sites, 15 of the next 30 sequences (50%) or 15 in the top 50 (30%) contain a three bp AT site flanked by a GC bp. This binding is weaker than the four bp AT sites, but significant enough to indicate that the X-ray observations of a GC interaction with the N-methylpiperazine represents the detection of this weaker binding to generic sequences represented by either GAAA or AAAG. Most prominent among these in the top 50 sequences of this assay are GATT (7, 14%) and GAAT (5, 10%). Unlike distamycin A, but like netropsin and DAPI, no site containing a GC bp central to a 5 bp AT-rich site is found in the top 50 sequences. Like the observations made with DAPI, the weaker binding of Hoechst 33258 with hairpins >100-150 (<20% fluorescent decrease) most likely represents that of the weaker intercalation. Most importantly, the intrinsic fluorescent enhancement upon Hoechst 33258 binding does not interfere with the assay.

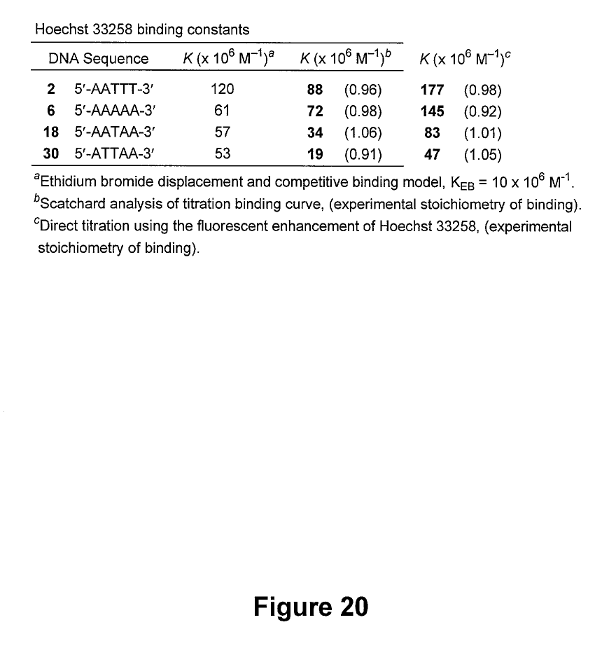

The binding constants for Hoechst 33258 were established for the four hairpins first examined with distamycin A (FIG. 20). Although no binding constants for these sequences have been disclosed, those established by footprinting (Matesoi, D.; et al. Biochem. Mol. Biol. lnt. 1996, 38, 123) or direct fluorescence titration (Loontiens, F. G.; et al. Biochemistry 1991, 30, 182; Quintana, J. R.; et al. Biochemistry 1991, 30, 10294) compare favorably with those established herein. Both the displacement of ethidium bromide and the measurement of a fluorescent decrease (excitation at 545 nm, emission at 595 nm) or the direct titration with Hoechst 33258 and the measurement of its fluorescent increase (excitation at 358 nm, emission at 454 nm) could be used to establish binding constants. Moreover, both titration binding curves confirmed the 1:1 stoichiometry of binding. Consistent with the trends observed with DAPI but slightly more pronounced with Hoechst 33258, the binding constants established by the ethidium bromide displacement were roughly and uniformly 2-fold weaker than those determined by direct titration. Thus, the former technique slightly underestimates the binding affinity.

Berenil. A member of the aromatic diamidine class of DNA binding agents, berenil, reversibly binds AT-rich duplex DNA. X-ray (Brown, D. G.; et al. EMBO J. 1990, 9, 1329; Brown, D. G.; et al. J. Mol. Biol. 1992, 226, 481) and NMR (Yoshida, M.; et al. Biochemistry, 1990, 29, 6585; Lane, A. N.; et al. Biochemistry, 1991, 30, 1372; Jenkins, T. C.; et al. Eur. J. Biochem. 1993, 213, 1175; Hu, S.; et al. Eur. J. Biochem. 1992, 204, 31) structures of the complex between berenil and d(CGCGAATTCGCG)₂ illustrate a 1:1 stoichiometry of binding with berenil residing in the minor groove of the AT tract. The terminal amidines were shown to engage in H-bonding with the terminal adenine N3 or thymine C2 carbonyls. The aromatic rings adopt an isohelical conformation and align parallel to the walls of the minor groove making close contacts with adenine. Footprinting studies established a sequence selectivity similar to that of netropsin and distamycin A with the highest affinity sites containing at least three contiguous AT bp (Laughton, C. A.; et al. Nucleic Acids Res. 1990, 18, 4479). In addition to binding in the minor groove and at higher concentrations, intercalative binding has also been observed (Pilch, D. S.; et al. Biochemistry 1995, 34, 9962).

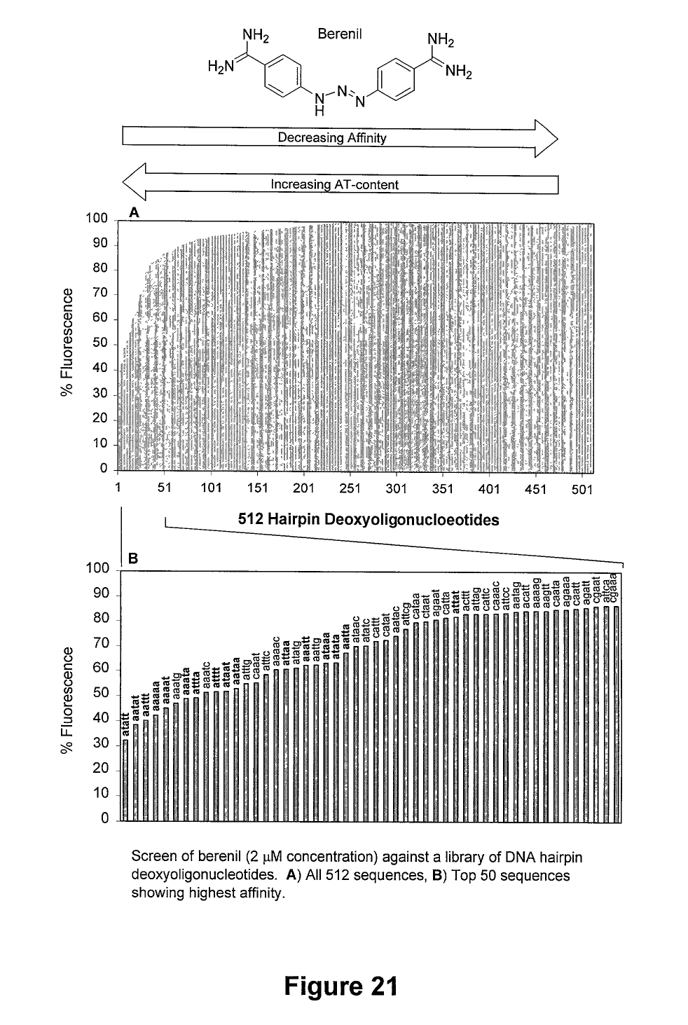

Its examination against the library of 512 hairpins revealed it to be a weaker DNA binding agent than the other compounds examined and that it exhibited the expected AT-rich binding selectivity (FIG. 21). Despite its small size, it still exhibited a 5>4>3 AT bp site selectivity and the tightest binding sequences all contained five, and to a lesser extent four, AT bp. In addition, 16/16 five bp AT sites (100%), 21/32 four bp AT sites (66%), and 12/80 three bp (15%) sites were found in the top 50 sequences. The binding is modest, even to the best sequences, and falls off rapidly such that only the top 20-30 sequences exhibit substantial binding at 2 μM.

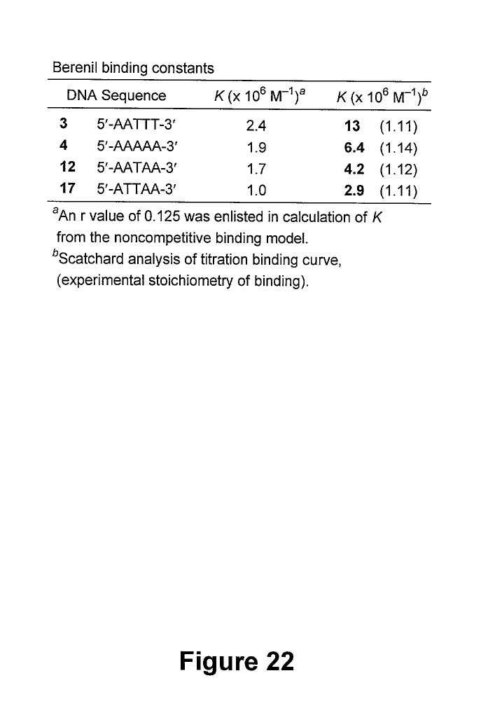

Enlisting the four hairpins first examined with distamycin A, the binding constants for berenil were established, FIG. 22. All the binding constants followed the expected trends from the ranked binding profile and all were lower than those of distamycin A or the remaining minor groove binders. The experimentally measured saturation of binding for berenil confirmed the expected 1:1 stoichiometry of binding to the selected hairpins.

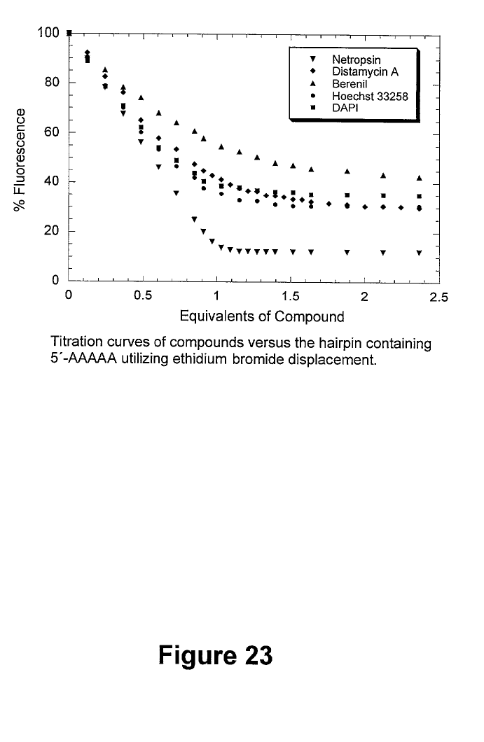

Displacement Stoichiometry. Initially, it was expected that the % fluorescence decrease at saturation binding would not only provide information on the stoichiometry of ethidium bromide displacement, but also on the binding site size of the compound. To a certain extent, this was observed with the saturation % fluorescence decrease following the expected order of distamycin A, Hoechst 33258≧DAPI>berenil corresponding roughly to the displacement of 3 and 2 equiv of ethidium bromide, respectively (FIG. 23). Inconsistent with expectations, netropsin displaced essentially all the ethidium bromide including that which would be expected to intercalate at the capping 5′-CGNNNNNC site. Although there is no present explanation for this behavior of netropsin, it does indicate that caution should be taken in comparing affinities when examining the bar graph profiles of two different compounds measured at a single concentration. That is, the % fluorescence decrease can reflect not only relative affinity, but also a different stoichiometry of ethidium bromide displacement. This limitation does not affect the comparisons made upon quantitative titration. Reflective of the titration curves, the binding constants for the hairpin containing AAAAA were established as follows: netropsin=347×10⁶ M⁻¹, Hoechst 33258=72×10⁶ M⁻¹, DAPI=50×10⁶ M⁻¹, distamycin A=17×10⁶ M⁻¹, and berenil=b 6.4×10⁶ M⁻¹.

In addition, the visual inspection of the titration binding curves for the five compounds indicate that netropsin, Hoechst 33258, and DAPI exhibit clean 1:1 binding with no evidence of a second binding event. By contrast, both distamycin A and berenil exhibit a weaker second or continued fluorescence decrease through the addition of 2 equiv of compound. For distamycin A, this most certainly represents the additional 2:1 side-by-side binding, whereas this may represent a second, lower affinity end-to-end binding for berenil.

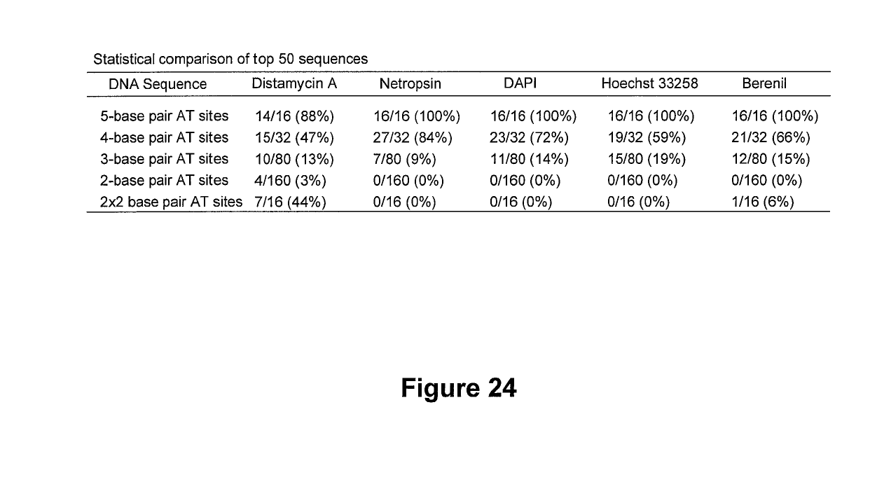

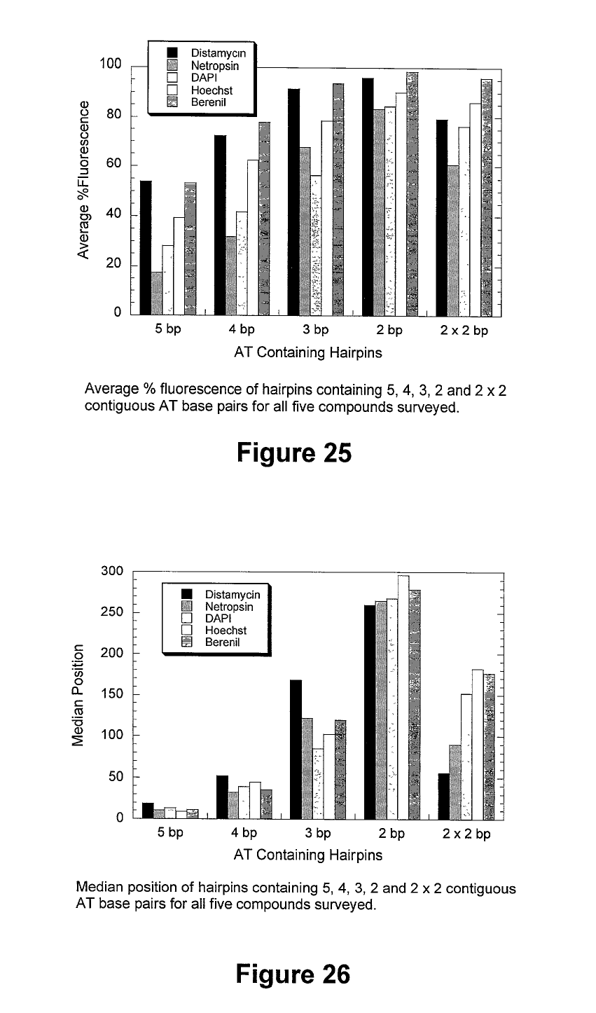

Selectivity of the Minor Groove Binding Agents. The intention of the studies detailed herein was not to redefine the well established selectivity of the minor groove binding agents. However, the results of the comparisons provide a few new insights that were not previously easily recognized. Analyzing either the average % fluorescence decrease for related sites (FIGS. 25 and 26) or evaluating the statistical site frequency occurrence (FIG. 24) in the top 50sequences offer approaches to comparing selectivities or recognizing distinctions. Netropsin is the most AT selective of the compounds examined and, by some accounts, distamycin A is the least AT selective. All exhibit tight binding to 5>4>3 bp AT sites. Presumably, this 5>4>3 bp AT site preference is related in part to the conformational characteristics of DNA where the longer AT-rich sites possess the narrower, deeper minor groove known to contribute substantially to the selective binding affinity. Arbitrarily selecting the top 50 sequences and analyzing the statistical site frequency, little distinction is observed for 5 bp AT sites although the frequency decreases slightly as netropsin, DAPI, Hoechst-33258, berenil (100%)>distamycin A (88%), FIG. 24. More significant was the frequency decrease observed for the 4 bp AT sites: netropsin (84%)>DAPI (72%)>berenil (66%)>Hoechst-33258 (59%)>distamycin A (47%), and the corresponding increase in 3 or 2 bp AT sites: distamycin A (27%)>Hoechst 33258 (19%)>berenil (15%)>DAPI (14%)>netropsin (9%). This represents the unique inclusion of AT-rich 5 bp sites containing a central GC base pair for distamycin A and the ability of Hoechst 33258 to bind 3 bp AT sites capped with a GC bp. It is suggested that this reflects (1) the larger five bp binding site requirement for distamycin A versus a four bp site for netropsin and the compensating ability for distamycin A to bind selected GC bp interrupted five bp AT-rich sites, (2) the ability for Hoechst 33258 to uniquely bind GATT/GAAT sites presumably via a G interaction with the terminal N-methylpiperazine, and (3) the smaller size of DAPI and berenil which more easily accommodates a three versus four bp AT site.

The preferences for a 5>4>3>2 bp AT site for all compounds studied and the ability for distamycin to bind a GC bp interrupted 5 bp AT site (2×2 bp) is also clear from the average % fluorescence decrease for the grouped sites (FIGS. 25 and 26). Thus, distamycin exhibits an affinity for the 2×2 bp AT sites that exceeds that of the of 3 bp AT sites and is only slightly weaker than that of the 4 bp AT sites. Netropsin shows a weaker preference for such 2×2 bp AT sites, none of which appear in the top 50 sequences (FIG. 24), whereas DAPI, Hoechst 33258, and berenil do not exhibit a similar unique affinity. An alternative way of examining the same data is to establish a median position in the 512 hairpin library for the grouped sequences (FIG. 26). This avoids some confusion in comparing the data due to the differences in the stoichiometry of ethidium bromide displacement (different % fluorescence decrease). Clearer in this plot of the data is the preference for distamycin binding to a 2×2 bp AT-rich site versus 3 bp AT site and the former is nearly as effective as binding to a 4 bp AT site. Netropsin shows a weaker ability to bind a 2×2 bp AT site which is comparable with a 3 bp AT site but much less effective than binding a 4 bp AT site. In contrast, DAPI, Hoechst 33258, and berenil exhibit a clear preference for a 3 bp AT site over a 2×2 bp AT site.

Thiazole Orange as an Alternative to Ethidium Bromide. The enhancement of fluorescence for the binding of ethidium bromide to duplex DNA is roughly 20-fold and varies from sequence to sequence. For the excitation and emission wavelengths used (545 nm and 595 nm, respectively), not only is the enhancement small and the sensitivity of the assay modest, but the fluorescence of some DNA binding agents may interfere at these wavelengths. As a consequence, use of an additional DNA intercalating dye, thiazole orange (TO) was examined. (Lee, L. G.; et al. Cytometry 1986, 7, 508). This DNA intercalating dye exhibits a different excitation and emission fluorescence (509 nm and 527 nm, respectively) (Nygren, J.; et al. Biopolymers 1998, 46, 39), displays a higher, but less sequence dependent affinity for DNA (Nygren, J.; et al. Biopolymers 1998, 46, 39), and produces a more intense fluorescence enhancement upon binding to duplex DNA (50-2000 vs 20-fold for ethidium bromide) (Nygren, J.; et al. Biopolymers 1998, 46, 39). The less sequence dependent DNA affinity suggests that thiazole orange should behave in a manner analogous to ethidium bromide, but that intrinsic errors derived from individual sequence variance may be further minimized. However, its greater affinity introduces errors potentially attributable to more effective competitive binding. More importantly, the sensitivity enhancement derived from the 10-100 fold increase in the fluorescence enhancement means the assay can be conducted with a more robust measurement signal and/or at even lower concentrations. The former increases the accuracy of the comparisons whereas the latter potentially reduces the reagent expense for conducting the assay. Presently, the ethidium bromide assay requires amounts of the 512 hairpins that cost $100/assay (20 cents/hairpin) that can be further reduced with the use of thiazole orange.

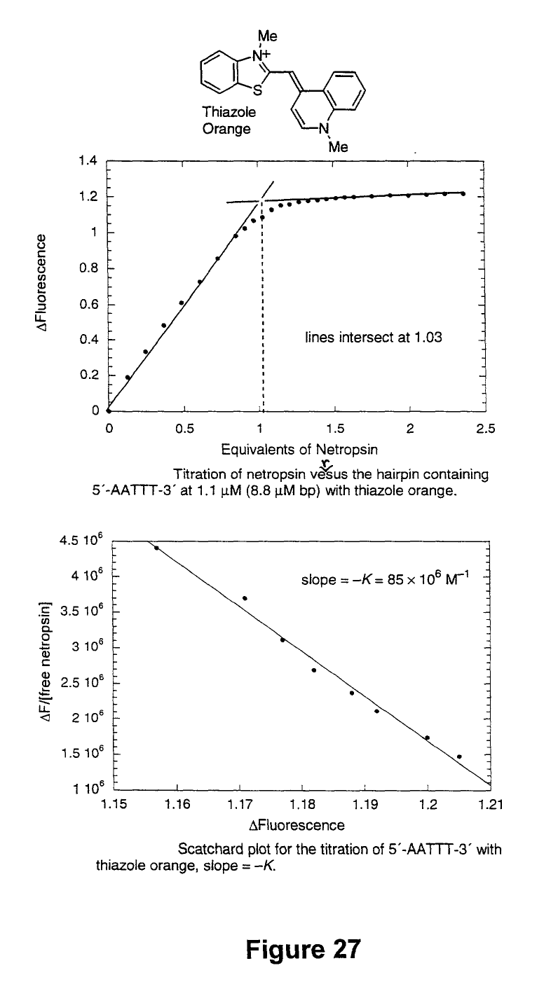

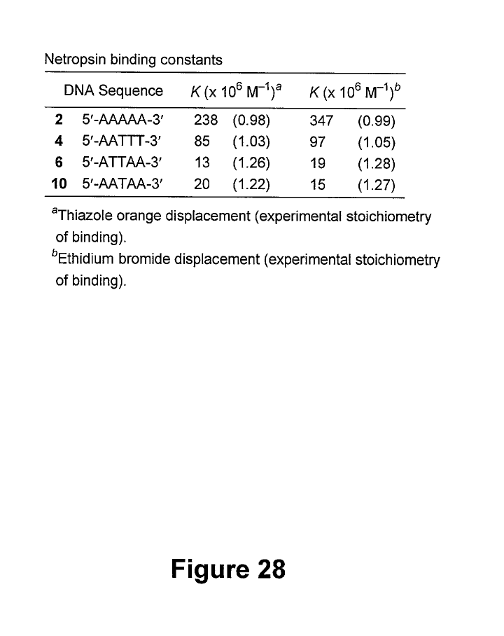

A preliminary examination of thiazole orange was conducted to assess the optimal assay concentration range, and to establish its affinity and stoichiometry of binding with representative hairpins (FIG. 10). Thiazole was utilized in both the 96-well format assay and in quantitative titrations for determining binding constants (FIGS. 27 and 28). These studies indicated that the use of thiazole orange permits the assay concentration to be reduced at least 2-4 fold reducing the amount and cost of the required hairpins accordingly. A representative example of the use of thiazole orange in the 96-well format assay is illustrated in FIG. 28 with the assay of netropsin against a 136-membered hairpin library containing all possible four bp sites. Netropsin exhibited the expected 4>3>2 bp AT selectivity. The mean rank of the ten available 4 bp AT sites was 7.3, whereas the mean positions for the 3 and 2 bp sites were 41.6 and 67.7, respectively. Moreover, the rank order binding across the full ensemble of 4 bp AT sites is consistent with that established in footprinting studies (Portugal, J.; Waring, M. J. Eur. J. Biochem. 1987, 167, 281; Portugal, J.; Waring, M. J. FEBS Lett. 1987, 225, 195; Abu-Daya, A.; et al. Nucleic Acids Res. 1995, 23, 3385; Abu-Daya, A.; Fox, K. R. Nucleic Acids Res. 1997, 25, 4962; Harshman, K. D.; Dervan, P. B. Nucleic Acids Res. 1985, 13, 4825; Van Dyke, M. W.; et al. Proc. Natl. Acad. Sci. USA 1982, 79, 5470; Ward, B.; et al. Biochemistry 1988, 27, 1198; Fish, E. L.; et al. Biochemistry 1988, 27, 6026; Kittler, L.; et al. J. Mol. Recognit. 1999, 12, 121). Notably, the highest affinity site is 5′-AAAA and the sequences 5′-TTAA, 5′-TATA, 3′-TAAA, and to a lesser extent 5′-ATAA, are among the lowest affinity 4 bp AT sites for netropsin and these same trends are observed in their rank order binding profile, i.e.: AAAA>ATAT, AATA, ATTA, AATT, AAAT>ATAA>TAAA, TATA, TTAA. Thus, although is has been less widely studied, the comparisons suggest thiazole orange is a superb screening alternative to ethidium bromide.

Consistent with past observations, the stoichiometry of binding and the Kₐₚₚ for thiazole orange for the four hairpins examined proved less sequence dependent than ethidium bromide, but it displayed higher binding constants (FIG. 10). The netropsin binding constants and stoichiometry of binding established using the displacement of thiazole orange proved to be in good agreement with those established with ethidium bromide (FIG. 28), albeit lower. The additional comparisons of binding constants established for DAPI and Hoechst 33258 illustrate these similarities and distinctions in the use of ethidium bromide and thiazole orange (FIG. 24). Binding constants for both DAPI and Hoechst 33258 were established by direct titration, the results of which can be used to assess the accuracy of the ethidium bromide or thiazole orange displacement titration results. In each case, the direct and indirect titration methods accurately reflected the trends observed in the 96-well format assay (rank order affinity). However, the intercalator displacement titrations systematically underestimated the binding constants. The constants established with ethidium bromide typically were within an acceptable factor of 2 or less of those established by direct titration, whereas the systematic derivation with thiazole orange was noticeably larger. Presumably, this reflects the greater competitive binding of thiazole orange, reducing the measured apparent binding constant. Interestingly, the sequence dependent variance in the binding affinity of ethidium bromide did not introduce errors of significance that were detectable. As such, the studies indicate that the binding constants established with ethidium bromide are preferred, and for most practical purposes, are comparable in both the trends and absolute magnitudes established by direct titration.

### Experimental Section

Ethidium Bromide Assay. 512-Member Deoxyoligonucleotide Library. Hairpin deoxyoligonucleotides were purchased from Genbase Inc. (The full set of 512 hairpin deoxyoligonucleotides may be purchased from GenBase, 6450 Lusk Blvd, Suite E 107, San Diego, Calif. 92121, telephone: 858-453-8879, e-mail genbase@aol.com.) as 1200 μM (bp) solutions in water and stored as stock solutions at −80° C. Prior to use, each deoxyoligonucleotide was diluted to 120μ in water and stored at 0° C. for no longer than two days. Each well of a Costar black 96-well plate was loaded with Tris buffer containing ethidium bromide (88 μL of a 0.68×10⁻⁵ solution in buffer (0.1 M Tris, 0.1 M NaCl, pH 8), 0.60×10⁻⁵ M ethidium bromide final concentration). To each well was added one hairpin deoxyoligonucleotide (10 μL, 1.2×10⁻⁵ M in DNA bp final concentration) followed by agent (2 μL of a 0.1 mM solution in water, 2.0×10⁻⁶ M final concentration). After incubation at 25° C. for 30 min, each well was read (average of 30 readings) on a Molecular Devices Spectra Max Gemini fluorescent plate reader (ex. 545 nm, em. 595 nm, cutoff filter at 590 nm) in duplicate with two control wells (no agent=100% fluorescence, no DNA=0% fluorescence). Fluorescence readings are reported as % fluorescence relative to the control wells. Fluorescence plate readers show a variability of ±10%, but surface effects (i.e bubbles, dust) may contribute to larger variations requiring a second set of measurements.

All other experiments described herein (i.e. compound concentration, overall assay concentration, ethidium bromide:DNA ratio, time variability, etc.) utilized this general procedure in a 96-well plate format.

Determination of Binding Constants. Ethidium Bromide/Thiazole Orange Displacement. A 3 mL quartz cuvette was loaded with Tris buffer (0.1 M Tris, 0.1 M, NaCl, pH 8) and ethidium bromide orthiazole orange (0.44×10⁻⁵ M final concentration). The fluorescence was measured (ex. 545 nm, em. 595 nm, EtBr) and normalized to 0% relative fluorescence. The hairpin deoxyoligonucleotide of interest was added (1.1 μM, 8.8 μM in bp final concentration), the fluorescence was measured again and normalized to 100% relative fluorescence. A solution of compound (2 μL, 0.1 mM in DMSO) was added and the fluorescence was measured following 5 min of incubation at 23° C. The addition of 2 μL aliquots was continued until the system reached saturation and the fluorescence remained constant with subsequent compound additions.

Direct Fluorescence Titration of DAPI, Hoechst 33258, Ethidium Bromide or Thiazole Orange. A 3 mL quartz cuvette was loaded with Tris buffer (0.1 M Tris, 0.1 M, NaCl, pH 8) and hairpin deoxyoligonucleotide (1.1 μM, 8.8 μM in bp final concentration). A solution of DAPI (excitation 372 nm; emission 454 nm; 2 μL, 0.1 mM in DMSO), Hoechst 33258 (excitation 358 nm; emission 454 nm; 2 μL, 0.1 mM in DMSO), ethidium bromide (excitation 545 nm; emission 595 nm; 2 μL, 0.5 mM in H₂O) or thiazole orange (excitation 509 nm; emission 527 nm; 2 μL, 0.5 mM in DMSO) was added and the fluorescence was measured following 5 min of incubation at 23° C. The addition of 2 μL aliquots was continued until the system reached saturation and the fluorescence remained constant with subsequent additions.

Scatchard Analysis of the Titration Curve. The AF was plotted versus molar equivalents of agent and the ΔFₛₐₜ was determined mathematically by solving the simultaneous equations representing the pre and post saturation regions of the titration curve. Utilizing equations (1)-(3) (FIG. 11), a Scatchard plot was generated where ΔF/[Free Agent] was plotted versus ΔF. The slope of the region immediately preceding complete saturation of the system provided −K.

### DETAILED DESCRIPTION OF FIGURES

FIG. 1 is a simplified illustration of the general procedure for the rapid DNA binding screen. The chosen DNA as homopolymers, heteropolymers or hairpin oligonucleotides is placed in 96 well plates. Upon treatment with ethidium bromide there is a large increase in fluorescence as ethidium bromide intercalates with the DNA. When a non-fluorescent DNA binding agent is added there is a percentage decrease in the fluorescence due to binding. The percentage decrease in fluorescence is proportional to the extent of DNA binding. This provides the relative DNA binding affinities and through quantitative titration that may be carried out later, an accurate, absolute binding constant is obtained.

FIG. 2 shows the hairpin DNA oligomer used in the binding affinity assay. A survey of distamycin A binding to all possible 5 base pair DNA sequences was conducted using a library of 512 of these hairpin DNA oligonucleotides containing all possible five base pair sequences of the general format 5′-GCXXXXXC-3′ with a 5-A loop. Although there are 1024 possible sequences containing 5 base pairs, two complementary sequences are contained in each hairpin differing only in their location relative to the position of the adenine loop making, for example, the sequence 5′-ATGCA equivalent to the sequence 5′-TGCAT as shown in the lower portion of the figure.

FIG. 3 shows the results of screening the 512-membered library of hairpin oligo-nucleotides using distamycin A. As expected, affinity increases with increasing AT content. The top sequences include the sites 5′-ATAA, 5′-AATT, 5′-AAAT, and 5′-AAAA and among the twenty hairpins showing the greatest decrease in % fluorescence, three four base-pair sequences occur most often: 5′-AATT, 5′-AAAT, 5′-AATA.

FIG. 4 is a bar graph showing the difference in change of fluorescence at two different compound concentrations with four different DNA sequences. The four DNA sequences are selected because they have much different affinities for distamycin A. For the assays conducted with a 1.5 μM concentration of the hairpin (12 μM in bp) 1 or 2 μM compound concentrations for assay met these criterion. Use of lower compound concentrations resulted in less discrimination between the sequences. This use of a near 1:1 ratio of compound to DNA provided the desired robust intensity of measure % fluorescence decrease suitable for a high throughput assay across a range of compounds with varied affinities.

FIG. 5 is a bar graph showing the results of a test designed to find the concentration of DNA required to provide a robust assay reading. The intention was to minimize the amount of DNA and reagents needed in a standard 96-well format (Costar black opaque, 100 μL assay volume) while maintaining a robust and reliable measurement. The hairpin deoxyoligonucleotide concentration for a high affinity sequence (AATTT) and low affinity sequence (AAACC) for distamycin A was varied from 0.375-3.0 μM (3-24 μM bp) in 0.1 M Tris, 0.1 M NaCl (pH 8) buffer maintaining a 1:2 ethidium bromide:base pair ratio and a constant ratio of distamycin A:hairpin. Concentrations of 1.5 μM hairpin (12 μM bp) or higher in a 100 μL volume maintained an acceptably constant reading to be reliable for both hairpins whereas those below 1.5 μM (12 μM bp) suffered variations too large to be considered useful.

FIG. 6 is a bar graph of a survey of concentration range for distamycin A versus a tight binding sequence (AATTT), and a modest binding sequence (AACCC). With the highest affinity AATTT sequence, concentrations as low as 0.5 μM and optimally 1 or 2 μM distamycin A performed well across the range of hairpin concentrations (3-24 μM). With the lower affinity AAACC sequence, concentrations of 2-8 μM (2-4 μM optimally) distamycin A performed well across the range of hairpin concentrations whereas the lower concentrations of 0.5-1 μM were not useful regardless of the hairpin concentration.

FIG. 7 is two bar graphs which show the results of the ethidium bromide:DNA ratio and the influence of DNA concentration on percent fluorescence. The upper bar graph shows the hairpin used in the experiment where distamycin A (2 μM) and DNA (AAATT) hairpin deoxynucleotide, 12 μM in base pair) concentrations were kept constant, while ethidium bromide concentration was varied. Measurements 1-4 were taken in succession after 40 minutes of incubation. Measurement 5 was taken after 2 h of incubation. Under conditions where the salt concentrations are physiologically relevant, little impact was observed when this ratio was varied over a range of 1:4 to 2:1 EtBr:bp.

The second bar graph shows why it is important in this experiment to tightly control the concentration of hairpin. Measurements conducted with hairpin concentrations lower than expected (e.g., 3 and 6 versus 12 μM bp) yield much larger than expected reductions in the % fluorescence, falsely indicating higher affinity binding. Similarly, measurements conducted with hairpin concentrations higher than expected (18 to 24 versus 12 μM) result in lower than expected reductions in the % fluorescence and underestimate the compound binding affinity.

FIG. 8 is a table showing the few absolute binding constants for distamycin A to short AT-rich sequences that have been published. The comparison of all those disclosed show the relative trend 5′-AATTT>AAAAA>AATAA>ATTAA (Rentzeperis, D.; et al. Biochemistry 1995, 34, 2937 and Wade, W. S.; Mrksich, M.; Dervan, P. B. Biochemistry 1993, 32, 11385). The ethidium bromide displacement assay revealed the same general trend and a quantitative titration measurement of binding constants with the hairpin oligo-nucleotides containing these sequences afforded binding constants that are not only consistent with the relative trend (FIG. 21), but also within a factor of 2-3 of all the absolute binding constants previously determined through calorimetry and footprinting (Table 4). Given that the DNA upon which the measurements were made is different, that the buffer conditions are not identical, and that entries 2-4 were derived from a close analog of distamycin A, all which may contribute to small discrepancies in the absolute binding constants, the ethidium bromide displacement titration assay appears to be remarkably accurate at reproducing absolute binding constants.

FIG. 9 is two plots. The first plot is of the linear region of the % fluorescence vs. distamycin A/DNA (base pairs). This graph can be extrapolated to estimate the number of distamycin molecules per length of DNA. Here the plot shows that about one molecule binds for every eight base pairs of DNA. The second plot shows the change in fluorescence versus the equivalents of EtBr. This graph yields the number of ethidium bromide molecules intercalated per hairpin. The linear regions are extrapolated to give a number of 3.3 molecules of ethidium bromide per hairpin.

FIG. 10 is a table showing the binding constants of ethidium bromide and thiazole orange. The first small table shows the wide variation in the binding constant of ethidium bromide to various polynucleotides (almost 60-fold difference between two different sequences) and no difference in binding constants with thiazole orange to three different polynucleotides. The second table shows essentially the same thing with the ethidium bromide having an almost 4-fold difference and thiazole orange having only a 1.3 fold difference. The variation in the DNA used in the first table is much greater than that used in the second table which explains the greater variation in the ethidium bromide constants.

FIG. 11 is a list of three equations used for a Scatchard analysis of titration binding curves. In cases where the binding stoichiometry is 1:1, binding constants may be established by Scatchard analysis. Equations 1-3 are used to establish the free agent concentration employed to generate a Scatchard plot from which binding constants may be determined. In these equations, [Free Agent]=concentration of free agent, [DNA]_{T}=total concentration of DNA, X=molar equivalents of agent versus DNA, DFₓ=change in fluorescence, DFₛₐₜ=change in fluorescence at the point where DNA is saturated with ligand.

FIG. 12 is two plots. The first plot shows the change in fluorescence versus equivalents of netropsin. This provides a titration curve from which the stoichiometry of binding can be derived. The second is a plot of DF/[Free Agent] versus DF which yields the a linear portion of the Scatchard plot and provides −K as the slow of this portion of the curve.

FIG. 13 shows a screen of netropsin against a library of DNA hairpin deoxynucleotides. Netropsin is a well characterized minor groove binder with high affinity for AT sites. Affinity increased with increasing AT content and it exhibited a 5>4>3 base pair binding selectivity. The top eight sequences and 13 of the top 15 sequences were five versus four base pair AT sites. All five base AT sequences were found in the top 33 sequences. Therefore, 16/16 five base pair AT sites were in the top 50 sequences (100%), 27/32 four base pair AT sites were in the top 50 sequences (84%), but only 7/80 three base pair AT sites were found in the tope 50 sequences (9%).

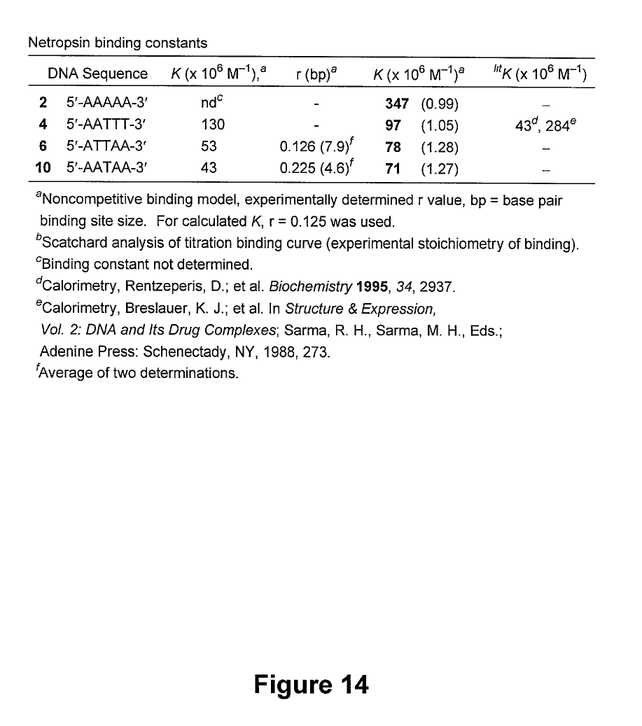

FIG. 14 is a table of netropsin binding constants. The netropsin binding constants were established for several hairpin sequences by quantitative titration using the four sequences first enlisted for distamycin A. The binding clearly reflects a 1:1 stoichiometry and the Scatchard analysis of the binding curves was good. The affinities of netropsin were approximately five fold greater than distamycin A.

FIG. 15 illustrates the screen of DAPI against a library of DNA hairpin deoxy-oligonucleotides. Plot A shows all 512 sequences of the library and plot B shows the 50 sequences showing the highest affinity. DAPI was examined against a set of 512 hairpin oligonucleotides at a 2 μM concentration and exhibited a high selectivity for a binding site containing at least three AT base pairs.. It is clear from the binding rank order that DAPI binds five and four base pair AT sites very effectively. Like netropsin, but unlike distamycin A, none of the top 50 sequences contain a central GC base pair central to a five base pair AT-rich site.

FIG. 16 is a table of the binding constants for DAPI determined for the four hairpin sequences first examined with distamycin A. The characteristic fluorescence enhancement of DAPI upon DNA binding could be monitored in a direct titration and used to independently establish binding constants and the 1:1 binding stoichiometry. The values obtained by this direct method closely match those obtained in the ethidium bromide displacement titration.

FIG. 17 is a plot of the titration of DAPI versus the hairpin containing 5′-ATTAA-3′ at 1.1 μM (8.8 μM bp) utilizing the inherent fluorescence of the DNA:DAPI complex. Three other sequences (AATTT, AAAAA, and AATAA) were tried and the plots gave the same 1:1 DAPI:DNA hairpin stoichiometry.

FIG. 18 is the Scatchard plot for the titration of 5′-ATTAA-3′ utilizing the inherent fluorescence of the DNA:DAPI complex=−K.

FIG. 19 is a screen of Hoechst 33258 against a library of DNA hairpin deoxy-oligonucleotides. The compound exhibited AT-rich binding when assayed against the library of 512 hairpin deoxyoligonucleotides. Its profile of selectivity lies somewhere between that of DAPI and distamycin A and this is clear from a simple comparison of the merged bar graphs of the 512 hairpin binding results.

FIG. 20 is a table of binding constants for Hoechst 33258 for four DNA sequences. Both the displacement of ethidium bromide and the measurement of a fluorescence decrease (excitation at 545 nm, emission at 595 nm) or the direct titration with Hoechst 33258 and the measurement of its fluorescence increase (excitation at 358 nm, emission at 454 nm) could be used to establish binding constants. Both the titration binding curves confirmed the stoichiometry of binding to be 1:1.

FIG. 21 is a screen of berenil against a library of DNA hairpin deoxyoligonucleotides. This screen revealed it to be a weaker DNA binding agent than the other minor groove binding agents examined and that it exhibited the expected AT-rich binding selectivity. Despite the small size of the molecule, it still exhibited a 5>4>3 AT base pair site selectivity and the tightest binding sequences all contained five, and to a lesser extent four, AT base pairs. In addition, 16/16 five base pair AT sites (100%), 21/32 four base pair AT sites (66%), and 12/80 three base pair sites (15%) were found in the top 50 sequences. The binding is modest, even to the best sequences, and falls off rapidly such that only the top 20-30 sequences exhibit substantial binding at 2 μM.

FIG. 22 is a table of berenil binding constants with the four sequences that have been used to test the other compounds. It shows the binding constant and the experimental stoichiometry of binding. The binding is much weaker with this compound than the previous compounds.

FIG. 23 is a graph of the titration curves for each of the five compounds tested with the hairpin containing 5′-AAAAA utilizing ethidium bromide displacement. The % fluorescence decrease at saturation binding would not only provide information on the stoichiometry of ethidium bromide displacement, but also on the binding site size of the compound. This is true to a certain extent as observed with the saturation % fluorescence decrease following the expected order of distamycin A, Hoechst 33258≧DAPI>berenil corresponding roughly to the displacement of 3 and 2 equivalents of ethidium bromide, respectively. Netropsin displaced essentially all the ethidium bromide including that which would be expected to intercalate at the capping 5′-CGXXXXXC site.

FIG. 24 is a table of statistical site frequency occurrence of the top 50sequences between the compounds. Netropsin is the most AT selective of the compounds examined and by some accounts, distamycin A is the least AT selective. All exhibit tight binding to 5>4>3 base pair AT sites. Arbitrarily selecting the top 50 sequences bound by each compound and analyzing the statistical site frequency, little distinction is observed for 5 base pair AT sites although the frequency decreases slightly as netropsin, DAPI, Hoechst-33258, berenil (100%)>distamycin A (88%). More significant was the frequency decrease observed for the 4 base pair AT sites: netropsin (84%)>DAPI (72%)>berenil (66%)>Hoechst-33258 (59%)>distamycin A (47%), and the corresponding increase in 3 or 2 base pair AT sites: distamycin A (27%)>Hoechst 33258 (19%)>berenil (15%)>DAPI (14%)>netropsin (9%).

FIG. 25 is a bar graph which illustrates the differing affinities for particular types of AT containing hairpins. From the graph, it is clear that the preference for a 5>4>3>2 base pair AT site holds for all the compounds studied.

FIG. 26 is a bar graph that is an alternative way to examine the same data as FIG. 48. A median position in the 512 hairpin library is established for the grouped sequences. Again, it is clear there is a preference for 5>4>3>2base pair AT sites. From the graph distamycin has a similar preference for GC bridged AT pairs as it has for four AT base pair sites. The other compounds have a clear preference for four base pair sites.

FIG. 27 is two plots. The first one shows the change in fluorescence versus the number of equivalents of added netropsin. The netropsin is displacing thiazole orange from the DNA. The structure of thiazole orange is given above the plot. The linear portions of this plot are long and allow for a clean extrapolation to 1.03 equivalents of netropsin. The second plot is a Scatchard plot which gives the binding constant for thiazole orange to this particular hairpin DNA.

FIG. 28 is a table giving netropsin binding constants to four different hairpin DNA oligonucleotides. The two different constants were obtained by displacing thiazole orange and ethidium bromide from the hairpin DNA with netropsin.

What is claimed is:

1. A process for determining DNA binding affinity and sequence selectivity of a test molecule, the process comprising the following steps:
Step A: measuring a fluorescent intercalator displacement of the test molecule with respect to each DNA binding reagent within an array of dna binding reagents, the array of DNA binding reagents including a plurality of hairpin deoxyoligonucleotides dissolved in an aqueous solvent together with a fluorescent DNA intercalator, each hairpin deoxyoligonucleotide within said plurality being physically separate from all other hairpin deoxyoligonucleotides within said plurality, said fluorescent DNA intercalator being displaceable from said hairpin deoxyoligonucleotides by the test molecule; and then
Step B: correlating the fluorescent intercalator displacement by the test molecule within the array of DNA bind reagents as determined in said step a for determining the DNA binding affinity and sequence selectivity of the test molecule.

2. A process according to claim 1 wherein each hairpin deoxyoligonucleotide includes a single strand hairpin section and a double strand section, the single strand hairpin section consisting of a non-variable sequence of unpaired deoxynucleotide bases, the double strand section including a variable sequence of paired deoxynucleotide bases, the variable sequence having a set length of between 4 and 14 deoxynucleotide bases, the variable sequence being known or identifiable, the double strand section being attached to the single strand hairpin section; said plurality of hairpin deoxyoligonucleotides including substantially all possible sequences within the set length of the variable sequence.

3. A process according to claim 1 wherein said Step A includes the following substeps:
Substep A(1): detecting and recording fluorescence for each DNA binding reagent within the array of DNA binding reagents; then
Substep A(2): combining an aliquot of the test molecule with each DNA binding reagent after said Step A; then
Substep A(3): detecting and recording fluorescence a second time for the array of DNA binding reagents after said Step B for determining fluorescent intercalator displacement.

4. A process according to claim 1 wherein said fluorescent DNA intercalator is selected from the group consisting of ethidium bromide and thiazole orange.

5. A process according to claim 1 wherein the array of DNA binding reagents is contained by an array of microtiter wells.

6. A process according to claim 1 wherein the set length is 4 and said plurality of hairpin deoxyoligonucleotides includes 136 hairpin deoxyoligonucleotides.

7. A process according to claim 1 wherein the set length is 5 and the plurality of hairpin deoxyoligonucleotides includes 512 isolated hairpin deoxyoligonucleotides.

8. A library employable for assaying DNA binding affinity and sequence selectivity, the library comprising:
a plurality of hairpin deoxyoligonucleotides, each hairpin deoxyoligonucleotide including
a single strand hairpin section consisting of a non-variable sequence of unpaired deoxynucleotide bases; and
a double strand section including a variable sequence of paired deoxynucleotide bases, the variable sequence having a set length of between 4 and 14 deoxynucleotide bases, the variable sequence being known or identifiable;
the double strand section being attached to the single strand hairpin section;
said plurality of hairpin deoxyoligonucleotides including substantially all possible sequences within the set length of the variable sequence;
each hairpin deoxyoligonucleotide within said plurality being physically separate from all other hairpin deoxyoligonucleotides within said plurality.

9. A library according to claim 8 wherein the set length is 4.

10. A library according to claim 9 wherein said plurality of hairpin deoxyoligonucleotides includes 136 hairpin deoxyoligonucleotides.

11. A library according to claim 8 wherein the set length is 5.

12. A library according to claim 11 wherein said plurality of hairpin deoxyoligonucleotides includes 512 isolated hairpin deoxyoligonucleotides.

13. An array of DNA binding reagents for determining DNA binding affinity and sequence selectivity of a test molecule, the array comprising:
a plurality of hairpin deoxyoligonucleotides, and
an aqueous solvent employable for use in a fluorescent intercalator displacement assay,
the hairpin deoxyoligonucleotides being dissolved in said aqueous solvent and having a concentration sufficient for use in the screening assay for DNA binding, each hairpin deoxyoligonucleotide within said plurality being physically separate from all other hairpin deoxyoligonucleotides within said plurality, each hairpin deoxyoligonucleotide including a single strand hairpin section and a double strand section, the single strand hairpin section consisting of a non-variable sequence of unpaired deoxynucleotide bases, the double strand section including a variable sequence of paired deoxynucleotide bases, the variable sequence having a set length of between 4 and 14 deoxynucleotide bases, the variable sequence being known or identifiable, the double strand section being attached to the single strand hairpin section;
said plurality of hairpin deoxyoligonucleotides including substantially all possible sequences within the set length of the variable sequence.

14. An array according to claim 13 further comprising:
a fluorescent DNA intercalator, said fluorescent DNA intercalator being intercalated into said hairpin deoxyoligonucleotides and being potentially displacable from said hairpin deoxyoligonucleotides by the test molecule, said fluorescent DNA intercalator being of a type that has altered fluorescent properties upon displacement said hairpin deoxyoligonucleotides.

15. An array according to claim 14 wherein said fluorescent DNA intercalator is selected from the group consisting of ethidium bromide and thiazole orange.

16. An array according to claim 14 wherein the set length is 4.

17. An array according to claim 16 wherein said plurality of hairpin deoxyoligonucleotides includes 136 hairpin deoxyoligonucleotides.

18. An array according to claim 14 wherein the set length is 5.

19. An array according to claim 18 wherein said plurality of hairpin deoxyoligonucleotides includes 512 isolated hairpin deoxyoligonucleotides.

20. An article for use in a fluorescent intercalator displacement assay for determining DNA binding affinity and sequence selectivity of a test molecule, the article comprising:
one or more microtiter plates, each microtiter plate having an array of microtiter wells, and
a plurality of hairpin deoxyoligonucleotides, each hairpin deoxyoligonucleotide being physically separate from all other hairpin deoxyoligonucleotides within said plurality and being individually contained within and corresponding to one of the microtiter wells, each hairpin deoxyoligonucleotide being present within its corresponding microtiter well with sufficient quantity for use in the fluorescent intercalator displacement assay, each hairpin deoxyoligonucleotide including a single strand hairpin section and a double strand section, the single strand hairpin section consisting of a non-variable sequence of unpaired deoxynucleotide bases, the double strand section including a variable sequence of paired deoxynucleotide bases, the variable sequence having a set length of between 4 and 14 deoxynucleotide bases, the variable sequence being known or identifiable, the double strand section being attached to the single strand hairpin section;
said plurality of hairpin deoxyoligonucleotides including substantially all possible sequences within the set length of the variable sequence;
said microtiter plates having a sufficient total number of microtiter wells for containing all of said plurality of hairpin deoxyoligonucleotides.

21. An article according to claim 20 further comprising:
a fluorescent DNA intercalator, said fluorescent DNA intercalator being intercalated into said hairpin deoxyoligonucleotides and being potentially displacable from said hairpin deoxyoligonucleotides by the test molecule, said fluorescent DNA intercalator being of a type that has altered fluorescent properties upon displacement from said hairpin deoxyoligonucleotides.

22. An article according to claim 21 wherein said fluorescent DNA intercalator is selected from the group consisting of ethidium bromide and thiazole orange.

23. An article according to claim 20 wherein the set length is 4, said plurality of hairpin deoxyoligonucleotides includes 136 hairpin deoxyoligonucleotides, and said microtiter plates having, all together, a total of at least 136 bmicrotiter wells.

24. An article according to claim 20 wherein the set length is 5, said plurality of hairpin deoxyoligonucleotides includes 512 hairpin deoxyoligonucleotides, and said microtiter plates having a total, all together, of at least 512 microtiter wells.
